(19) United States
(12) Patent Application Publication
(10) Pub. No.: US 2020/0386751 A1
(43) Pub. Date: Dec. 10, 2020

(54) FIELD EFFECT TRANSISTOR SENSOR DETECTION ASSAYS AND SYSTEMS AND METHODS OF MAKING AND USING SAME

(71) Applicant: iNanoBio, Inc., Scottsdale, AZ (US)

(72) Inventor: Bharath Takulapalli, Fremont, CA (US)

(73) Assignee: iNanoBio, Inc., Scottsdale, AZ (US)

(21) Appl. No.: 16/767,575

(22) PCT Filed: Nov. 29, 2018
(86) PCT No.: PCT/US2018/063170

§ 371 Date: May 27, 2020

Related U.S. Application Data
(60) Provisional application No. 62/591,209, filed on Nov. 28, 2017.

(51) Int. Cl. G01N 33/543; G01N 27/74

(52) U.S. Cl. CPC G01N 33/54373; G01N 27/745

(57) ABSTRACT

Devices, systems and methods for detecting target analyte molecules or particles in a sample and in some cases, determining a measure of the concentration of the molecules or particles in the fluid sample are disclosed.

### RELATED APPLICATIONS

This application claims the benefit of U.S. application Ser. No. 62/591,209, entitled ENZYME LINKED FIELD EFFECT TRANSISTOR SENSOR ASSAYS FOR AMPLIFIED BIOMARKER DETECTION, METHODS OF MAKING AND USING, and filed Nov. 28, 2017, the contents of which are hereby incorporated herein by reference.

### FIELD OF DISCLOSURE

The present disclosure generally relates to systems and methods for detecting target analyte molecules or particles in a fluid sample and in some cases, determining a measure of the concentration of the molecules or particles in the fluid sample.

### BACKGROUND OF THE DISCLOSURE

Methods and systems that are able to quickly and accurately detect and, in certain cases, quantify a target analyte molecule or particle in a sample are desirable for a variety of applications. Such systems and/or methods could be employed in many areas, such as academic and industrial research, environmental assessment, food safety, medical diagnosis, and detection of chemical, biological and/or radiological warfare agents. Desired features of such techniques can include specificity, speed, and sensitivity.

Most current techniques for quantifying low levels of analyte molecules in a sample use amplification procedures to increase the number of reporter molecules in order to be able to provide a measurable signal. For example, such processes include enzyme-linked immunosorbent assays (ELISA) for amplifying the signal in antibody-based assays, as well as the polymerase chain reaction (PCR) for amplifying target DNA strands in DNA-based assays. A more sensitive but indirect protein target amplification technique, called immunoPCR (see Sano, T.; Smith, C. L.; Cantor, C. R. Science 1992, 258, 120-122), makes use of oligonucleotide markers, which can subsequently be amplified using PCR and detected using a DNA hybridization assay (see Nam, J. M.; Thaxton, C. S.; Mirkin, C. A. Science 2003; 301, 1884-1886; Niemeyer, C. M.; Adler, M.; Pignataro, B.; Lenhert, S.; Gao, S.; Chi, L. F.; Fuchs, H.; Blohm, D. Nucleic Acids Research 1999, 27, 4553-4561; and Zhou, H.; Fisher, R. J.; Papas, T. S. Nucleic Acids Research 1993, 21, 6038-6039). While the immuno-PCR method permits ultra low-level protein detection, it is a complex assay procedure, and can be prone to false-positive signal generation (see Niemeyer, C. M.; Adler, M.; Wacker, R. Trends in Biotechnology 2005, 23, 208-216).

One disadvantage of typical known methods and/or systems for accurately detecting and, optionally, quantifying low concentrations of a particular analyte in solution is that they are based on ensemble responses in which many analyte molecules give rise to a measured signal. Most detection schemes require that a large number of molecules are present in the ensemble for the aggregate signal to be above the detection threshold. This disadvantage limits the sensitivity of most detection techniques and/or the dynamic range (i.e., the range of concentrations that can be detected). Many of the known methods and techniques are further plagued with problems of non-specific binding, which is the binding of analyte molecules/particles to be detected or reporter species non-specifically to sites other than those expected. This can lead to an increase in the background signal, and therefore limits the lowest concentration that may be accurately or reproducibly detected.

Accordingly, improved methods for detecting and, optionally, quantifying analyte molecules or particles, especially in samples where such molecules or particles are present at very low concentration are desired.

### SUMMARY OF THE DISCLOSURE

This summary is provided to introduce a selection of concepts in a simplified form. These concepts are described in further detail in the detailed description of example embodiments below. This summary is not intended to necessarily identify key features or essential features of the claimed subject matter, nor is it intended to be used to limit the scope of the claimed subject matter.

As set forth in more detail below, the present disclosure relates to systems and methods for detecting (e.g., target) analyte molecules or particles in a fluid sample, and in some cases, determining a measure of the concentration of the molecules or particles in the fluid sample. The measure of the concentration of the molecules or particles in the fluid sample can be determined by, for example, quantifying the sensor response.

In accordance with at least one embodiment of the disclosure, a device for detecting a target analyte includes a sensor device comprising a surface exposed to an environment, a capture agent on or near the surface, the capture agent configured to selectively bind to a target analyte, optionally, a primary binding agent that binds to the target-analyte, a reporter enzyme conjugate that binds to the primary binding agent or to the target analyte, and an enzyme-substrate that undergoes biochemical reaction in presence of reporter enzyme to produce enzyme reaction products. As set forth in more detail below, the sensor device can produce a signal by detecting a change in an electrical property (e.g., a charge and/or a potential) of the environment or magnetic property of the environment or electrical property of the sensor surface or a mechanical property of the surface due to the production of the enzyme reaction products. The sensor device can be or include, for example, a field effect transistor (FET) sensor or a fully depleted exponentially coupled (FDEC) FET sensor. The target-analyte can include, for example, a molecule or biomarker or ionic species or cell or particle in a test medium. In accordance with exemplary aspects, the capture agent can be immobilized on or near the surface. In accordance with further examples, the enzyme reaction products comprise ions. The enzyme reaction products can bind directly to the surface. The enzyme reaction products can bind directly to the sensor surface or alternately bind to the sensor reference electrode or alternately bind to the sensor counter electrode or bind to active surface near the sensor (the binding can change a response of the sensor). The sensor device can be present in an array format comprising a plurality of sensor devices, with a unique capture agent immobilized on or near each of the sensor devices. Additionally or alternatively, the sensor device can be present in an array format comprising a plurality of sensor devices, with a unique capture agent immobilized on or near multiple sensors in the array. The sensor device can be present in an array format comprising a plurality of sensor devices to detect a multiplicity of target analytes. In accordance with exemplary aspects, the enzyme reaction products can also be detected using fluorescence or luminescence or other optical detection methods. Sensor device produces a signal due to enzyme reaction products that can also be detected in addition, optionally, using optical detection methods.

In accordance with at least one other embodiment of the disclosure, a device for detecting a target analyte includes a sensor substrate comprising a sensor device, a complimentary substrate comprising a capture agent to selectively bind to a target analyte, optionally, a primary binding agent that binds to the target-analyte, a reporter enzyme conjugate that binds to the primary binding agent or to the target analyte, and an enzyme-substrate that undergoes biochemical reaction in presence of reporter enzyme to produce enzyme reaction products. The sensor device can produce a signal by detecting a change in an electrical property of the environment or magnetic property of the environment or electrical property of the sensor surface or a mechanical property of the surface due to the production of the enzyme reaction products. The complimentary substrate can include an array of capture-agents. The sensor substrate comprises an array of sensor devices. Further, the sensor substrate comprising the array of sensor devices can be aligned/overlaid, brought close to or in contact with, the complimentary substrate comprising a matching array of capture-agents, to create a microfluidic channel for fluidic flow between the sensor substrate and the complimentary substrate. The sensor substrate and complementary substrate may have pillars or wells or patterns or microfluidic channels or other physical features. The flow can initiate a reporter enzyme reaction that results in enzyme-reaction-products. The complimentary substrate can include multiple capture agents, with a unique capture agent immobilized at each spot of a plurality of spots. Additionally or alternatively, the complimentary substrate can include epitope-tag fusion-proteins that are immobilized on surface using anti-epitope binding agents, wherein the fusion-proteins may be expressed in situ in an array format. The protein immobilized on complementary substrate may be wild type proteins, mutations of proteins, post translationally modified proteins, abnormal proteins, peptides, poly-peptides, denatured proteins, isoforms. In situ protein arrays may be produced and immobilized using methods such as NAPPA (Nucleic Acid-Programmable Protein Array) or isolated protein capture (IPC or contra capture or cover capture of version of NAPPA) or other in situ or ex situ protein production methods.

In accordance with at least other embodiment of the disclosure, a method for detecting a target analyte includes: providing a sensor device comprising a surface, providing a capture-agent on or near the surface, exposing the surface to an environment comprising a target analyte, optionally providing an primary binding agent that binds to the target analyte, providing a reporter enzyme conjugate that binds to a primary binding agent or to the target analyte, providing an enzyme-substrate that undergoes biochemical reaction in presence of the reporter enzyme to produce enzyme reaction products, and using the sensor device, producing a signal by detecting a change in an electrical property of the environment or a mechanical property of the surface due to the production of the enzyme reaction products. The sensor device can be, for example, a field effect transistor (FET) sensor or a fully depleted exponentially coupled (FDEC) FET sensor.

In accordance with at least another embodiment of the disclosure, a method for detecting a target analyte includes: providing a sensor device comprising a surface, providing a capture-agent on or near the surface, exposing the surface to an environment comprising a target analyte, optionally providing an primary binding agent that binds to the target analyte, providing an enzyme-substrate conjugate that binds to a primary binding agent or to the target analyte, providing a reporter-enzyme that undergoes biochemical reaction in presence of the enzyme-substrate to produce enzyme reaction products, and using the sensor device, producing a signal by detecting a change in an electrical property of the environment or a mechanical property of the surface due to the production of the enzyme reaction products. The sensor device can be, for example, a field effect transistor (FET) sensor or a fully depleted exponentially coupled (FDEC) FET sensor. Further, sensor signal producing a signal by detecting binding of enzyme reaction products on sensor surface.

In accordance with at least another embodiment of the disclosure, a method for enzyme linked sensor assay includes: providing a sensor device comprising a surface, optimally providing a capture-agent on or near the surface, the capture agent configured to bind one of either enzyme-substrate-conjugate or reporter-enzyme-conjugate that are complementary; immobilizing one of enzyme-substrate-conjugate or reporter-enzyme-conjugate immobilized on or near the sensor surface, directly or using capture agent, exposing the surface to an environment comprising the other of complementary reporter-enzyme or enzyme-substrate that undergoes biochemical reaction in presence immobilized conjugate and other reaction components to produce enzyme reaction products, and using the sensor device, producing a signal by detecting a change in an electrical property of the environment or a mechanical property of the surface due to the production of the enzyme reaction products. The sensor device can be, for example, a field effect transistor (FET) sensor or a fully depleted exponentially coupled (FDEC) FET sensor. Further, sensor signal producing a signal by detecting binding of enzyme reaction products on sensor surface.

The subject matter of the present disclosure can include, in some cases, interrelated products, alternative solutions to a particular problem, and/or a plurality of different uses of one or more systems and/or articles.

### BRIEF DESCRIPTION OF THE DRAWING FIGURES

A more complete understanding of exemplary embodiments of the present disclosure can be derived by referring to the detailed description and claims when considered in connection with the following illustrative figures.

FIG. 1 illustrates standard ELISA (enzyme-linked immunosorbent assay) formats.

FIG. 2 illustrates exemplary sensors suitable for use in accordance with various examples of the disclosure.

FIG. 3 illustrates an example of a direct enzyme-sensor linked assay in accordance with examples of the disclosure.

FIG. 4 illustrates an example of an indirect enzyme-sensor linked assay in accordance with examples of the disclosure.

FIG. 5 illustrates an example of a sandwich enzyme sensor linked assay in accordance with examples of the disclosure.

FIG. 6 illustrates an example of a sensor, a capture probe, a target analyte, a signal probe, and a reporter enzyme in accordance with examples of the disclosure.

FIG. 7 illustrates a device in accordance with examples of the disclosure in which the reporter enzyme conjugate is replaced with DNA or RNA strand conjugate, which then binds to the target analyte directly or via binding to a primary binding agent.

FIG. 8 illustrates a device in accordance with examples of the disclosure, where the capture agent is a DNA probe that is immobilized on or near the sensor device surface.

FIGS. 9-16 illustrate methods of forming and using various devices in accordance with various embodiments of the disclosure. FIG. 16 illustrates an assay in accordance with examples of the disclosure.

FIGS. 17(A) and (B) illustrate FDEC sensor detection of kinase enzyme activity initiated by flow or addition of enzyme substrate or co-factor. FIG. 17(C) illustrates FDEC sensor detection of inhibition of kinase enzyme activity in the presence of a drug molecule, as example case of drug discovery screening or drug resistance detection application. FIG. 17(D) illustrates FDEC sensor detection of Tau enzyme-substrate de-phosphorylation by phosphatase enzyme.

FIG. 18 illustrates a linked sensor assay (or ELTAA) of present invention detecting bacterial cell presence in a test medium, resulting in sensor response in accordance with exemplary embodiments of the disclosure.

It will be appreciated that elements in the figures are illustrated for simplicity and clarity and have not necessarily been drawn to scale. For example, the dimensions of some of the elements in the figures may be exaggerated relative to other elements to help to improve the understanding of illustrated embodiments of the present disclosure.

### DETAILED DESCRIPTION OF EMBODIMENTS OF THE INVENTION

Although certain embodiments and examples are disclosed below, it will be understood by those in the art that the invention extends beyond the specifically disclosed embodiments and/or uses of the invention and obvious modifications and equivalents thereof. Thus, it is intended that the scope of the invention disclosed should not be limited by the particular disclosed embodiments described below. The illustrations presented herein are not meant to necessarily be actual views of any particular material, structure, or device, but may be idealized representations that are used to describe embodiments of the disclosure.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to,” words using the singular or plural number also include the plural or singular number, respectively. Additionally, the words “herein,” “above” and “below” and words of similar import, when used in this application, shall refer to this application as a whole and not to any particular portions of this application. As used herein, the singular forms “a,” “an” and “the” include plural referents unless the context clearly dictates otherwise. “And” as used herein is interchangeably used with “or” unless expressly stated otherwise.

The description of exemplary embodiments of the disclosure provided below is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize.

Target analyte (used interchangeably with ‘target molecule’ or ‘target species’ or ‘biomarker’) as used here is something that is detected in a ‘test sample’ and represents in exemplary applications one or more of, but not limited to: biomolecule, organic molecule, inorganic molecule, ions, micro particle, nano particle, cell, vesicle, exosome, protein, antigen, dna, rna, peptides, enzymes, cytokines, hormones, growth factors, enzyme co factors, antibodies, membrane proteins, cell surface receptors, bacterial, pathogens, virus, fungus, eukaryotic cells, prokaryotic cells, lipids, metabolites, carbohydrates, sugars, glycans, PNA (peptide nucleic acid), biopolymers, drug molecules, organic or inorganic species, tissue, organs, organelles.

Test sample (used interchangeably with ‘test medium’) as used here is a test substance in which presence of one or more of target analytes is being tested, and represents one or more of, but not limited to: blood, saliva, bio sample, urine, stool, sputum, aqueous solution, organic solution, fluid, gas, liquid, tissue sample, tissue lysate, serum, plasma, pathogen growth medium, bacterial medium, chemical liquid, chemical mixture, aspirate, nose aspirate, lung aspirate, organ aspirate, fetal fluid, amniotic fluid.

Capture agent as used here is an agent or moiety or species or surface that binds/captures the test analyte. Non limiting examples of capture agents are: antibody, antigen, dna, rna, complementary oligomer, lipids, cell surface receptor, aptamer, glycans, beads, particles, magnetic particles, organic or inorganic molecule or surface, hydrophilic or hydrophobic surface, a micro or nano structured surface, nano porous or meso porous surface.

In one exemplary case, capture-agent can be a antibody and corresponding target-analyte can be antigen; in another example capture-agent can be an antigen and corresponding target-analyte can be an antibody; in another example capture-agent can be a DNA or RNA strand and corresponding target-analyte can be a complementary DNA or RNA strand; in another example capture-agent may be an aptamer and corresponding target-analyte can be a protein; in another example capture-agent may be a protein and corresponding target-analyte can be another protein that binds to it.

Capture agent can be provided in one or more of, but not limited to, exemplary configurations: (1) capture agent is provided/immobilized/printed/coated on the same surface or physical-substrate as the sensor, in the vicinity or proximity of the sensor (2) capture agent is provided/immobilized/printed/coated directly on the sensor surface, covering part of the sensor surface or whole of the sensor surface (3) capture agent is provided/immobilized/printed/coated on a second surface, that is immobilized surface is a second surface not same as the sensor surface, which may comprise one or more of, but not limited to, a second surface/physical-substrate or beads or particles or magnetic particles or nano particles, and the second surface (immobilized surface) is brought into proximity or vicinity or into contact with the first surface that has sensors. The words proximity and vicinity and near are used interchangeably and have the same meaning. In some exemplary applications proximity implies less than few hundred microns. In other exemplary applications proximity implies less than few microns. In other exemplary applications proximity implies less than few hundred nanometers. In other exemplary applications proximity implies less than few millimeters.

Sensor device (used interchangeably with ‘biosensor’ or ‘chemical sensor’ or ‘ion sensor’): One non-limiting example is field effect transistor sensor. Another non-limiting example is, FDEC (fully depleted exponentially coupled) FET sensor. Alternately, as used here, ‘sensor device’ represents one or more of, but not limited to: FET (field effect transistor) sensor, FDEC (fully depleted exponentially coupled) FET sensor, field effect sensor, charge sensor, potential sensor, workfunction sensor, GMR sensor, graphene sensor, molybdenum disulfide sensor, ISFET sensors, BJT (bipolar junction transistor) sensors, transistor sensors, capacitive sensors, resistive sensors, conductive sensors, electrochemical sensors, plasmonic sensors, SPR sensors, amperometric sensors, voltammetric sensors, carbon nanotube sensor, nanowire sensors, nanotube sensors, III-V based sensors, GaS sensors, electron spin sensor, GaN sensor, avalanche diode sensor, electron or hole tunneling sensor, flexible sensor, quantum wire sensor, GMR (giant magneto resistance) sensor, ion sensor, quartz crystal microbalance sensors, MEMs sensors, NEMs sensors, tuning fork sensors, piezo sensors, mechanical sensors. An exemplary sensor device is described in U.S. Pat. No. 9,170,228, issued Oct. 27, 2015, the contents of which are hereby incorporated herein by reference.

In one exemplary application, the sensor assay of present invention linked to FET sensors or FDEC FET sensors is termed Enzyme Linked Transistor Amplified Activity assay (ELTAA).

Sensor devices in general can be a single sensor or a device within an array of device sensors, or an array of nested array of sensors, either on a surface or a physical-substrate or embedded in wells or microwells or nanowells or pico wells or femto wells.

Reporter enzyme is an enzyme that catalyzes reactions of substrates in the test medium to produce products. Reporter enzyme may be ALP or other phosphatase, SRC or other kinase, HRP enzyme, etc. Non-limiting examples of reporter enzymes are kinases, proteases, DNAses, ubiquitination enzymes, phosphatases, oxidases, reductases, polymerases, hydrolases, lyases, transferases, isomerases, ligases, oxidoreductases, Glucosidases, Glycoside hydrolases, glycases, dehydrogenases, enolases, Secretases, synthases, Endonucleases, exonucleases, lipases, oxygenases, cellulases, cyclases, esterases.

Non-limiting examples of reporter enzyme products include: enzyme reaction products such as ions, ionic species, molecules, organic molecules, proteins, post translational modified substrates, epigenetically marked substrates, non-ionic molecules, proteins, peptides, amino acids, oligomers, nucleotides, glycans, sugars, lipids. In exemplary application, enzyme reaction products are ions such as but not limited to H+, Pi (phosphate ion), PPi (pyrophosphate) etc. A non-limiting example of reporter enzymes is alkaline phosphatase with its respective substrates, that releases ions as catalytic products. Another non-limiting example of reporter enzyme is HRP with thionine dye substrate that releases ion products.

Primary binding agent: is a molecule or species that binds to target analyte. In one non-limiting exemplary application this may be an antibody or a DNA oligomer or an antigen or a particle. In an exemplary application, capture agent and the complementary binding agent may bind the target analyte at different location or epitopes on target analyte. In one exemplary application the primary binding agent may be called primary tag.

Reporter-enzyme-conjugate: is a biomolecule conjugated with reporter enzyme, wherein the biomolecule binds to the primary-binding-agent. In one exemplary application the reporter enzyme conjugate may be called a secondary tag.

In one non-limiting exemplary application reporter-enzyme may be bound directly to complementary-binding-agent that binds to target-analyte (may be termed direct assays). In another non-limiting exemplary application tag-conjugated-reporter-enzyme may be used, where the tag binds to the complementary-binding-agent which in-turn binds to the target-analyte (may be termed indirect assays).

Capture agent can bind to test analyte with specificity or selectivity ranging from very high to very low, with binding strengths or affinities ranging from very high to very low. Methods of present disclosure can be used to detect presence or absence of target analyte in a test sample and/or to quantify the amount of target analyte present. Target analyte can be detected using the methods of present invention to as low as single molecule detection or single analyte detection.

Enzyme Substrate: enzyme substrate is a biomolecule or chemical upon which an enzyme acts, in presence of co-factors where required. Reporter enzyme catalyzes biochemical reactions involving the substrate, in the presence of other co-factors, buffers, reaction components. The Reporter-enzyme catalyzed biochemical reactions with enzyme-substrate as reactant results in enzyme-reaction-products.

In a non-limiting example, reporter enzyme catalyzes enzyme-substrates to release catalytic reaction products that include ions which interact with or bind to sensor to result in detection signal. In another non-limiting example, reporter enzyme catalyzes substrate to release products that include non-ionic species which interact/bind with sensor to result in sensor detection signal. In another non-limiting example, products of reporter enzyme substrate reaction comprise one or more of (a) reaction product that can be detected by sensor (b) reaction product that can be detected by light based spectroscopy methods, like fluorescence, luminescence, calorimetry, etc., both detections done either sequentially or simultaneously or in parallel or a combination.

Immobilized surface or immobilization surface: in one non-limiting example, immobilization surface is where capture agent and/or analyte and/or complementary binding molecule and/or conjugated reporter enzyme are bound or immobilized. In one non-limiting example, the immobilized surface can be on the same surface or physical-substrate as the sensor devices. In another non-limiting example, the immobilized surface is a different surface/physical-substrate, and the immobilized surface is brought in proximity or vicinity of a sensor surface. As used here, terms sensor substrate and complementary substrate refer to solid material substrates or objects. As used here, enzyme substrate refers to reactant chemical and biomolecules corresponding to an enzyme, where the enzyme catalyzes the enzyme substrates to enzyme-reaction-products which could be ions or other molecules.

As used here, near or proximity or vicinity represents one or more of, but not limited to, exemplary distances from the sensor surface: less than 100 micron distance from sensor surface, less than 50 micron distance from sensor surface, less than 25 micron distance from sensor surface, less than 10 micron distance from sensor surface, less than 5 micron distance from sensor surface, less than 1 micron distance from sensor surface, less than 0.5 micron distance from sensor surface, less than 1000 micron distance from sensor surface, less than 500 micron distance from sensor surface, or less than 250 micron distance from sensor surface. The distance may be greater than zero and less than any of these values.

The chemical and bio sensor described in the present invention can be used in exemplary applications for one or more of, but not limited to: (i) detect presence of target analyte in a test sample (ii) detect production or release or consumption of target analyte in a test sample (iii) detect addition or removal of target analyte to/from the test sample.

A non-limiting exemplary embodiment of a test instrument, where the test instrument and components may be applied for biomarker detection or disease diagnosis or prognosis or monitoring, using enzyme linked sensor array or ELTAA of present invention. In one non-limiting exemplary embodiment the test instrument comprises FET ion sensor technology or FDEC FET sensor technology. The test instrument may have small form factor, may be a desktop instrument, may be a hand held instrument, may be a hand portable instrument. In one non-limiting exemplary embodiment the test instrument may comprise of components including but not limited to: (i) sensor array chip, that may comprise array of sensor devices or array of nested sensor array devices. The sensor chip may be reusable after use or may be disposable after use; (ii) one of either reporter-enzymes or enzyme-substrates, and other enzyme reaction components, are provided on or near sensor device surface. The instrument may additionally comprise, as a non-limiting example, a second complementary substrate that has array of capture agents matching the sensor array on sensor chip, wherein the complementary substrate has immobilized reporter-enzymes or enzyme-substrates, in array format (iii) a microfluidic cartridge that comprises or is integrated with microfluidic channels, valves, pumps, actuators (iv) blister packs comprising solutions, buffers, reaction components, co-factors, complementary enzyme-substrate or reporter-enzyme either in a well or a isolated area or in a blister pack (v) electronic circuit such as, but not limited, to an analog integrated circuit or application specific integrated circuit, comprising AD converters, FPGAs, processors, memories, which is used for detection of sensor signal response, additionally in a multiplexed format, additionally in real time (vi) a outer box that integrates all these components of the instrument, and may have an external user interface, and may have digital or analogic interface for communicating with external instruments of computers or mobile devices (vii) a port for introducing test sample into instrument which may comprise target analytes to be detected (viii) computational algorithms and approaches that analyze sensor data and that may additionally provide inference on disease stage and future disease progress (ix) a slot for extracting the sensor array chip and/or may have additionally a slot for microfluidic cartridge and/or other component (x) components for display of data.

A device or enzyme sensor assay (or ELTAA) of present disclosure, and a test instrument based on the assays of current invention, may find applications in academic and industrial research, life sciences and biotechnology research, early disease detection, disease diagnosis, prognosis and post therapeutic monitoring, drug discovery, target identification, phenotypic screening, drug screening, disease pathway discovery, detection of clinical outcome measures, disease biomarker discovery, biomarker detection, personalized therapeutic development, precision medicine, preemptive diagnostics, pathogen detection, environmental assessment, food safety, medical diagnosis, veterinary diagnosis, agriculture and detection of chemical, biological and/or radiological warfare agents.

Methods included in references “Enzyme Linked Immunosorbent Assay (ELISA), John Crowther, Molecular Biomethods Handbook pp 657-682” and “Immuno-PCR: An ultrasensitive immunoassay for biomolecular detection, Anal Chim Acta. 2016 Mar. 3, 910:12-24”, are incorporated herein by reference, to the extent such methods do not conflict with present disclosure.

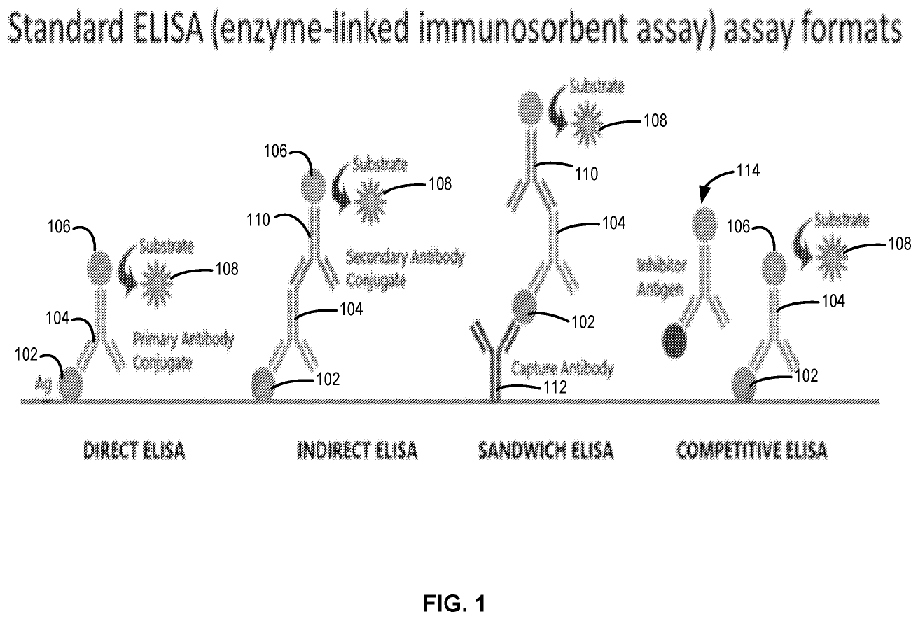

FIG. 1 illustrates various examples of ELISA, namely direct ELISA, indirect ELISA, sandwich ELISA, and competitive ELISA, which can be used in connection with various embodiments of the disclosure. With direct ELISA, a target molecule 102 can bind to a primary binding agent or biomolecule conjugate 104, which can bind to a reporter enzyme 106. As discussed in more detail below, a enzyme-substrate can undergo a biochemical reaction in presence of reporter enzyme 106 to produce enzyme reaction products 108. With Indirect ELISA, a secondary conjugate molecule 110 can be interposed between primary binding agent 104 and reporter enzyme 106. With sandwich ELISA, a capture agent 112 can bind to target molecule 102—e.g., to bind target molecule 102 to a substrate. Finally, in competitive ELISA, an inhibitor antigen can compete for binding to a limited amount of labeled antibody or antigen.

With typical ELISA, a reporter enzyme conjugated to primary or secondary antibody and bound/immobilized/complexed to the target analyte, can be assayed in the presence of enzyme substrate and the resulting fluorescence or luminescence or other optical signal is detected using light or UV or fluorescence or luminescence spectroscopies.

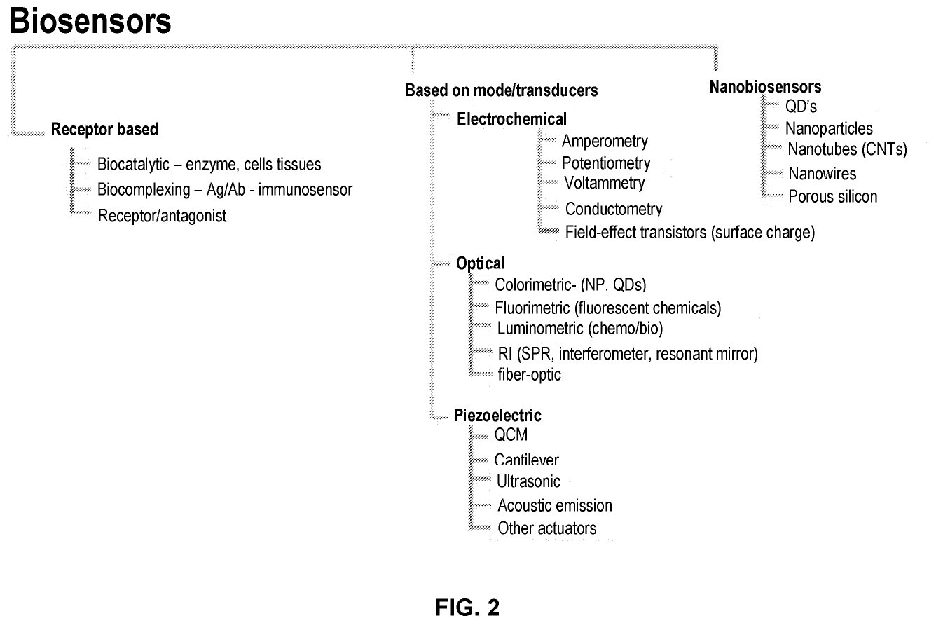

In contrast and in accordance with exemplary methods of present disclosure, a “reporter enzyme substrate catalytic reaction product” system can be selected, such that one or more of the reporter enzyme reaction products can be selectively detected by a chemical sensor and/or ion sensor and/or biosensor that is placed in the vicinity or proximity of the immobilized enzyme. Non-limiting exemplary chemical sensors and/or ion sensors and/or biosensors suitable for use in accordance with various examples of the disclosure are illustrated in FIG. 2.

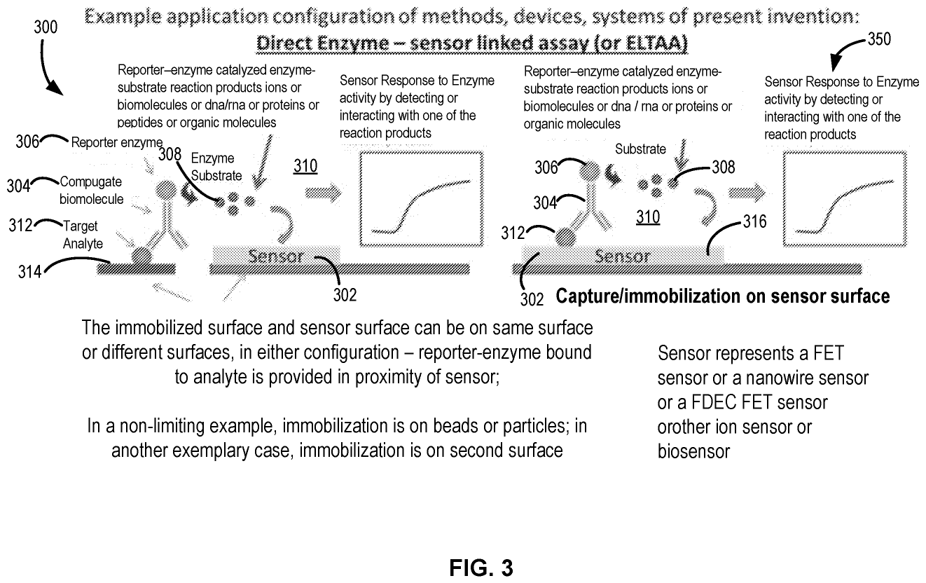

FIGS. 3-8 illustrate exemplary devices in accordance with various embodiments of the disclosure. Although a target analyte or probe is illustrated in the examples, devices can initially not include such target analytes, probes, probes or other target material. Such target material can be added during use of the exemplary devices.

FIG. 3 illustrates examples of a direct enzyme-sensor linked assay or device 300 and 350 in accordance with examples of the disclosure. Devices 300, 350 include a sensor device 302, a conjugate biomolecule 304, a reporter enzyme 306, and an enzyme substrate. A reaction between the reporter enzyme 306 and the enzyme substrate produces reaction products 308 and a change in environment 310, which in turn, can be detected by sensor device 302. Devices 300 and 305 are similar, except a target analyte 312 is bond directly to sensor device 302 surface 316 in device 350, whereas in device 300, target analyte 312 is bound to a surface 314 separated from sensor device surface 316. Surface 314 and surface 316 could form the same surface of a solid-substrate. Sensor device 302, and other sensor devices described herein can be or include an FDEC FET sensor or FET sensor, such as a sensor disclosed in U.S. Pat. No. 9,170,228, the contents of which, to the extent such contents do not conflict with the present disclosure, are hereby incorporated herein by reference.

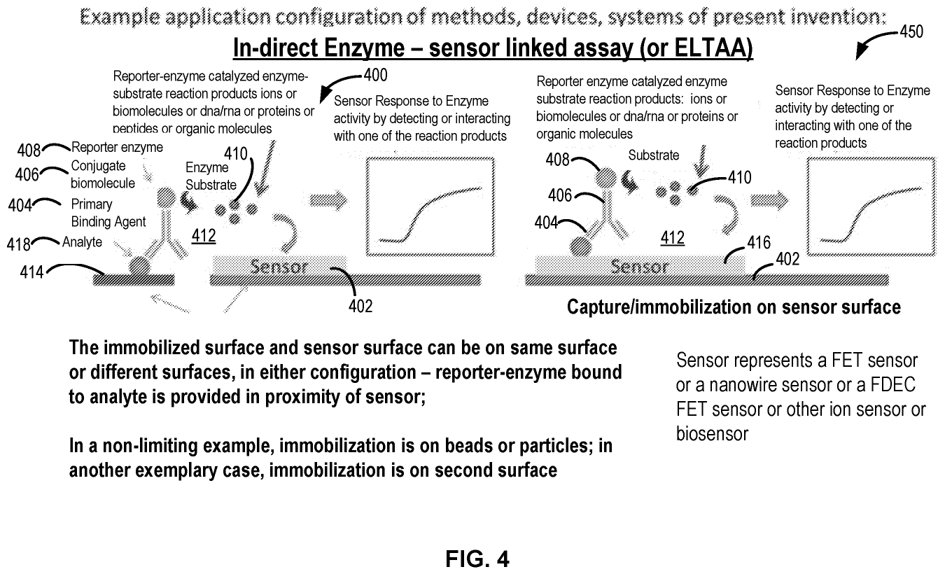

FIG. 4 illustrates device 400, 450 in accordance with examples of the disclosure. Devices 400, 450 include a sensor device 402, a primary binding agent 404, a conjugate binding agent 406, a reporter enzyme 408, and an enzyme substrate. A reaction between the reporter enzyme 408 and the enzyme substrate produces reaction products 410 and a change in environment 412, which in turn, can be detected by sensor device 402. Target analyte 418 can be bound to sensor device surface 416 or another surface 414 separated from the sensor device surface (e.g., on the same substrate as the sensor device).

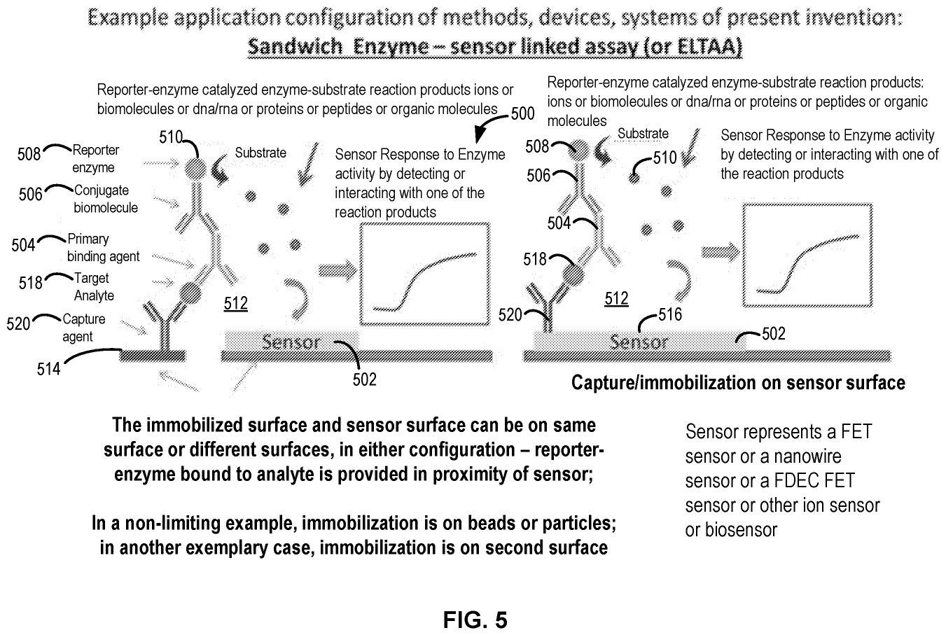

FIG. 5 illustrates devices 500, 550 with a sandwich enzyme sensor linked assay in accordance with examples of the disclosure. Devices 500, 550 are similar to devices described above, except device 500, 550 include a capture agent 520, in addition to a sensor device 502, a primary binding agent 504, a conjugate binding agent 506, a reporter enzyme 508, and an enzyme substrate. A reaction between the reporter enzyme 508 and the enzyme substrate produces reaction products 510 and a change in environment 512, which in turn, can be detected by sensor device 502. Target analyte 518 can be bound to sensor device surface 516 or another surface 514 separated from the sensor device surface (e.g., on the same substrate as the sensor device).

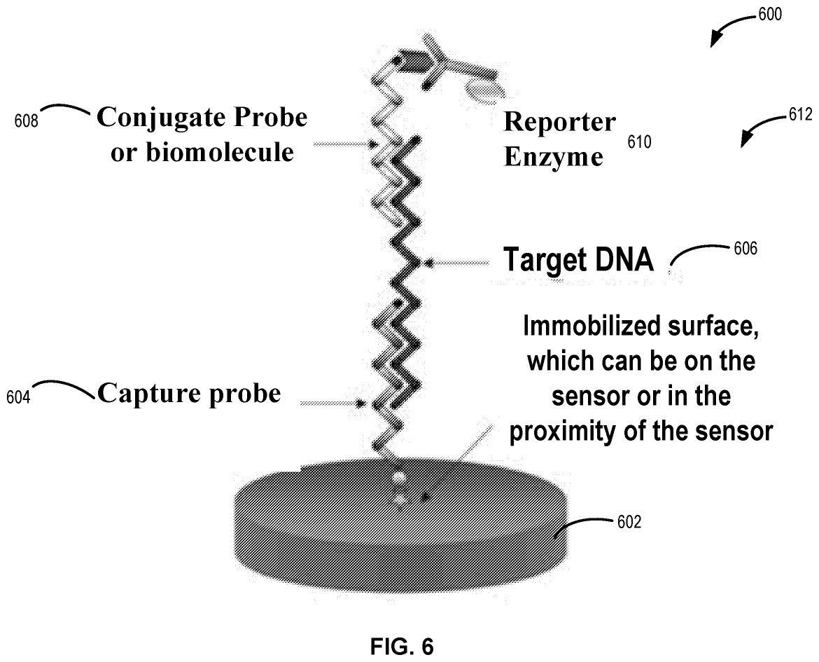

FIG. 6 illustrates a portion of a device 600 suitable for DNA analysis. Device 600 can be or include a DNA probe that is immobilized on or near the sensor surface. In this case, a target agent is a DNA oligomer in test medium that binds to DNA probe. The reporter enzyme conjugate is a DNA strand that is conjugated to the reporter enzyme, and binds to the target DNA. DNA DNA binding may be via complementary sequence portions/regions in the pairing strands, which could bind via hybridization. Introducing flow or enzyme-substrate or co-factor will initiate the reporter enzyme reaction that produces enzyme reaction products resulting in a sensor signal.

In the illustrated example, device 600 includes a surface (e.g., of a sensor device) 602, a capture probe or agent 604, a target analyte 606, a conjugate probe or biomarker 608, and a reporter enzyme 610 in accordance with examples of the disclosure. The reporter enzyme 610 can be, for example, any ELISA reporter enzyme or PCR polymerase enzyme as used in immuno-PCR method. In the case of reporter enzyme being PCR polymerase, detection of the reaction products by the sensor can be ions released, or amplified DNA produced, or other catalytic PCR reaction products. In another example PCR polymerase can be replaced with RT-PCR enzyme that produces cDNA from RNA. Device 600 can include any sensor as described herein. Device 600 can also include an enzyme substrate.

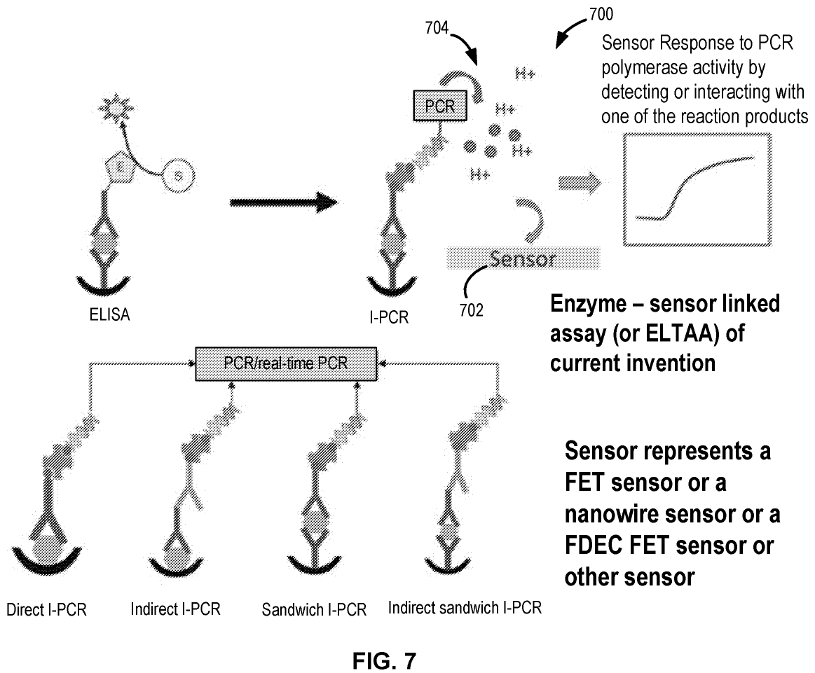

FIG. 7 illustrates a device 700 suitable for PCR in accordance with examples of the disclosure. In this case, the reporter enzyme conjugate is replaced with a DNA or RNA strand conjugate, which then binds to the target analyte directly or via binding to a primary binding agent. Enzymes such as polymerases, transcriptases or reverse-transcriptases are introduced into reaction mixture with other components, along with primers, to initiate strand synthesis reaction. DNA/RNA strand will be acted upon by the enzyme, resulting in release of ions as reactions products, which are detected by the FET sensor to indicate presence of DNA/RNA conjugate bound target analyte.

Device 700 includes a sensor device 702 and PCR products, which can be formed using various PCT techniques, including those illustrated in FIG. 7.

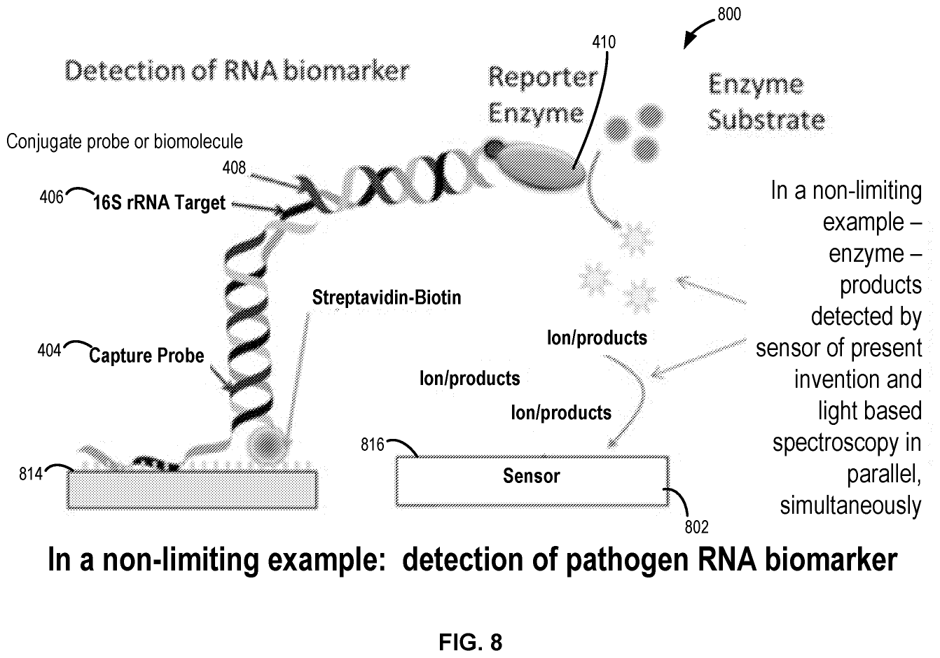

FIG. 8 illustrates a device 800 suitable for detection of RNA in accordance with yet additional embodiments of the disclosure. Device 800 includes a sensor 802, a capture agent 804 (e.g., is a DNA probe) that is immobilized on or near the surface 814 and/or 816. Exemplary target agent 406 is a RNA oligomer in test medium that binds to DNA probe 404. A reporter enzyme conjugate 408 can be a DNA strand that is conjugated to the reporter enzyme 410, and binds to the Target RNA. DNA RNA binding may be via complementary sequence portions/regions in the pairing strands, which could bind via hybridization. Introducing flow or enzyme-substrate or co-factor will initiate the reporter enzyme reaction that produces enzyme reaction products resulting in a sensor signal.

Solid-state sensors, such as sensor devices 302, 402, 502, 702, 802, and other sensor devices described herein, can be used in a wide variety of applications. For example, chemical solid-state sensors may be used for real-time analysis of chemical mixtures in both continuous and discrete sampling modes. Similarly, biological sensors/sensor devices can be used to detect biological agents and hazards and radiation sensors can be used to detect types and amounts of radiation.

The sensors/sensor devices can be used to detect a single component in a complex mixture, such as a toxic molecule in ambient atmosphere, analyze multiple components in a composition, or perform characterization and quality assessment of complex mixtures—e.g., as used to characterize odors, tastes, smells, etc., by pattern recognition methods using array-based sensors.

Typical solid-state sensors generally include a detection or receptor element and signal transduction means. The receptor layer interacts with the target specie(s), capture agent, or the like—e.g., by physical absorption or physisorption, chemisorption, microencapsulation, or the like. The transducer converts a change at the receptor surface (e.g., in an environment as described above) into a measurable electrical signal. The signal transduction, or coupling of signal between the receptor and the transducer may be linear, nonlinear, logarithmic or exponential in relation. The coupling relation between the two elements generally determines the sensitivity of the device.

A variety of signal transducer elements, such as potentiometric sensors, amperometric sensors, conductometric sensors, field effect transistor (FET) based sensors, optical sensors, thermal sensors, gravimetric or piezo-electric sensors, and the like, have been developed. FET devices may be particularly desirable because the FET devices exhibit relatively fast and sensitive signal transduction, are relatively easy to use, and are relatively easy to integrate with other sensor components.

In the case of FET devices, the metal gate of the field effect transistor device can be either replaced or coated with a sensitive thin film, insulator or membrane, which acts as the signal detection element. The FET devices work on the general principal of detecting shifts in localized electric potential due to interactions at the device surface. The FET device transduces a detection event into an electrical signal by way of change in the conductance of the channel region leading to a change in the drain current. The FET device may be operated as a sensor either by biasing the device with constant gate voltage and measuring the change in the current or by detecting the change in gate voltage required to maintain a constant current.

Metal-oxide-semiconductor FET (MOSFET) type sensors are often operated in inversion mode, where inversion current is established in the semiconductor channel by biasing the metal gate of the MOSFET. In these devices, target molecule binding (directly or indirectly) at the sensitive thin film or a change in radiation level modulates the minority charge carrier density in the inversion channel. Hence, inversion current in a bulk p-type MOSFET decreases upon addition of negative charge to the device surface.

Although such devices and transducer elements have been shown to work for some sensing applications, the non-FET devices are relatively bulky and expensive, and the typical FET-based devices may be relatively unstable and exhibit relatively low sensitivity.

The present invention provides an improved solid-state sensor for use as a sensor devices as described herein for detection of biological and chemical species and for radiation detection. More particularly, the exemplary embodiments of the disclosure provide a field-effect transistor (FET) including quantum wire(s) or nanowire(s), which operates as a fully-depleted exponentially-coupled (FDEC) sensor. As discussed in greater detail below, a threshold voltage or channel conductance of the sensor is manipulated as sensed ionic or biological, chemical, or radioactive species are detected, causing an exponential change in channel current.

The exponential change of channel current of the sensors of the present invention is in an opposite direction compared to that of typical FET sensors, and increases in n-channel type devices upon detection of species having excess electron charge or negative charge. Such an exponential response makes the sensors of the present invention more sensitive for qualitative and quantitative analysis.

In contrast to the present invention, the prior art teaches inversion-based FET devices applied for chemical sensing, with variations of device structure in a manner where addition of negative charge to the surface of an n-channel FET causes a decrease in inversion channel conductance (or drain current decrease), and addition of positive charge causes an increase in inversion channel conductance; and where, in a p-channel FET, addition of negative charge to the surface of the device causes an increase in channel conductance (or drain current increase) and addition of positive charge causes a decrease in channel conductance. Such responses of device structures is in opposite direction to the device of the present invention in this application. As noted above, in accordance with various embodiments of the invention, the addition of negative charge to the surface of an n-channel inversion based FET device in accordance with the present invention increases the inversion channel conductance, and addition of positive charge to the surface decreases the inversion channel conductance, while addition of negative charge to the surface of a p-channel inversion based device decreases the inversion channel conductance and addition of positive charge to the surface, increases the inversion channel conductance.

Channel region of FET sensor device or FDEC FET sensor device may include the structures or pores throughout the region thickness, or the structures and/or pores may be formed on a top surface. In accordance with various examples of the invention, top surface of FET sensor includes micro-pores, meso-pores, nano-pores, or macro-pores i.e., the pore diameter size may range from about 3 Å to 100 microns or from about 10 Å to about 10 mm. In accordance with additional embodiments of the invention, a FET sensor channel region includes structures, such as nano-structures or micro structures, having dimensions ranging from about 3 Å to about 100 microns. A width of each of the structures can be from about 10 Angstroms to about 10 millimeters. The structures may include, for example, nano or micro scale pillars of square, circular, triangular, hexagonal, any suitable cross section. The structures may also include micro or meso or nano porous structure superimposed on a nano or micro pattern in relief or recess.

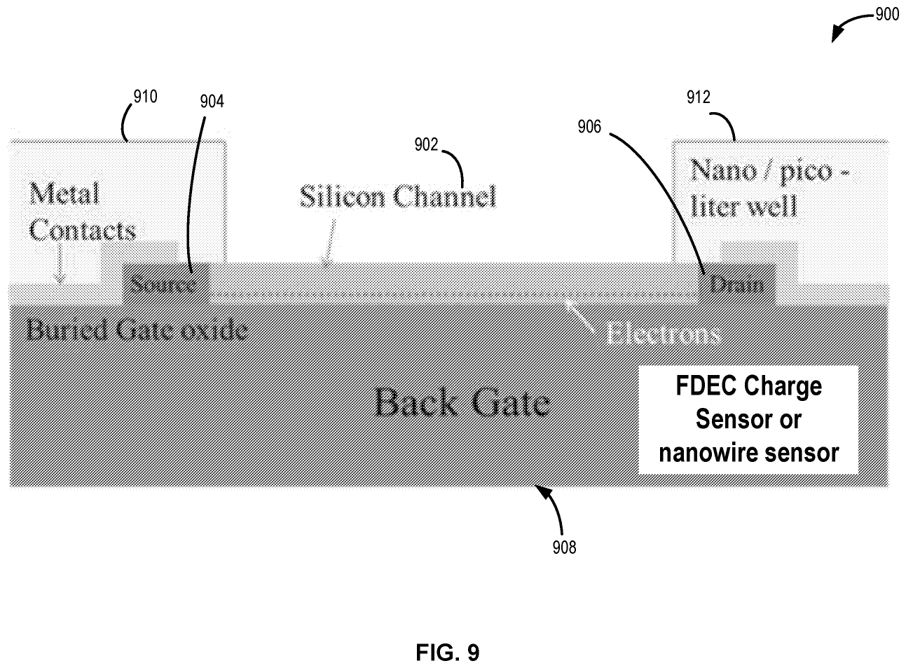

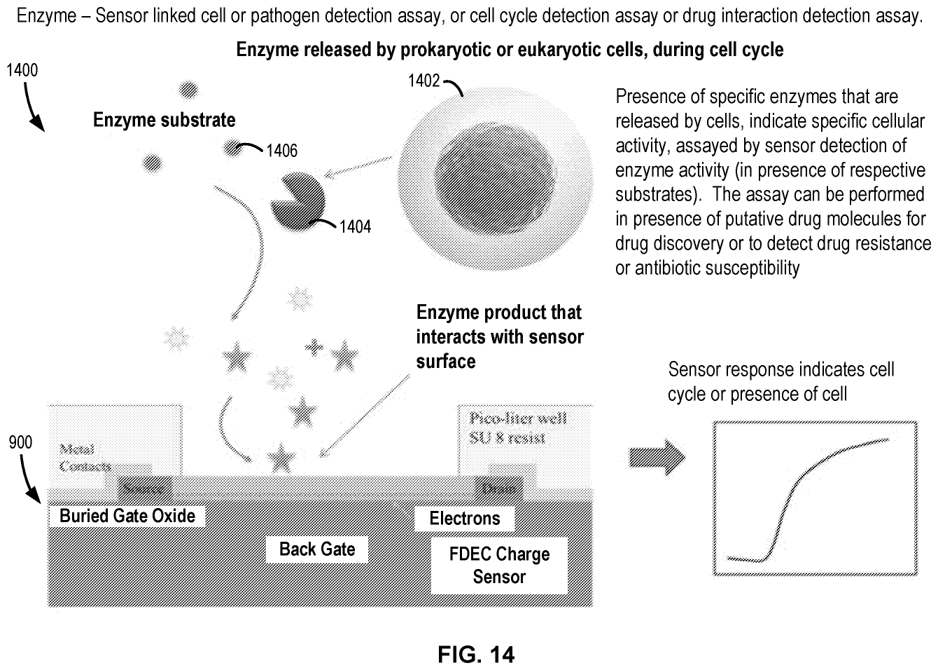

Turning now to FIGS. 9-16 and 18, non-limiting exemplary devices, sensor devices, and methods of using the devices and sensor devices are illustrated. FIGS. 9-13 illustrate examples and steps in the formation and use of the exemplary devices. As set forth in more detail below, FIGS. 14-16 and 18 illustrate devices and assays suitable for detection of cell activity.

1. Provide a Sensor

FIG. 9 illustrates an exemplary sensor device 900 in accordance with exemplary embodiments of the disclosure. In the illustrated example, sensor device 900 is an FDEC (fully depleted exponentially coupled) FET (field effect transistor). However the sensor shown can be replaced with any other non-optical sensor, such as but not limited to: FET (field effect transistor) sensor, field effect sensor, charge sensor, potential sensor, workfunction sensor, GMR sensor, graphene sensor, molybdenum disulfide sensor, ISFET sensors, BJT (bipolar junction transistor) sensors, transistor sensors, capacitive sensors, resistive sensors, conductive sensors, electrochemical sensors, plasmonic sensors, SPR sensors, amperometric sensors, voltammetric sensors, carbon nanotube sensor, nanowire sensors, nanotube sensors, III-V based sensors, GaS sensors, GaN sensor, p-n junction sensor, diode sensor, avalanche diode sensor, electron or hole tunneling sensor, flexible sensor, quantum wire sensor, GMR (giant magneto resistance) sensor, orion sensor.

Sensor device 900 includes a channel 902, a source region 904, a drain region 906, and a back gate 908. Sensor device 900 can also include metal contact 910, 912 (inside the insulating film that opens window on sensor surface). As noted above, channel 902 can be patterned and/or include features.

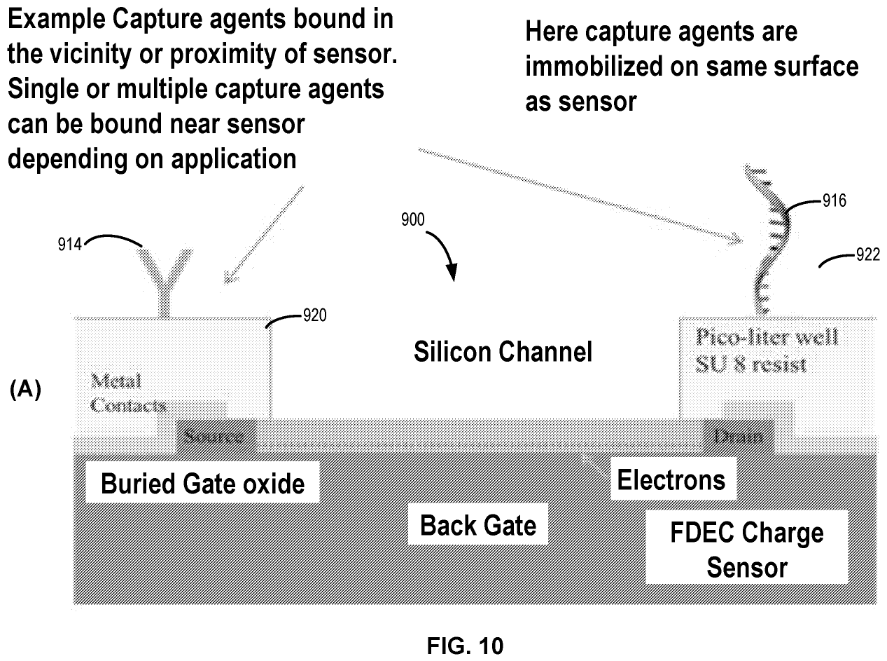

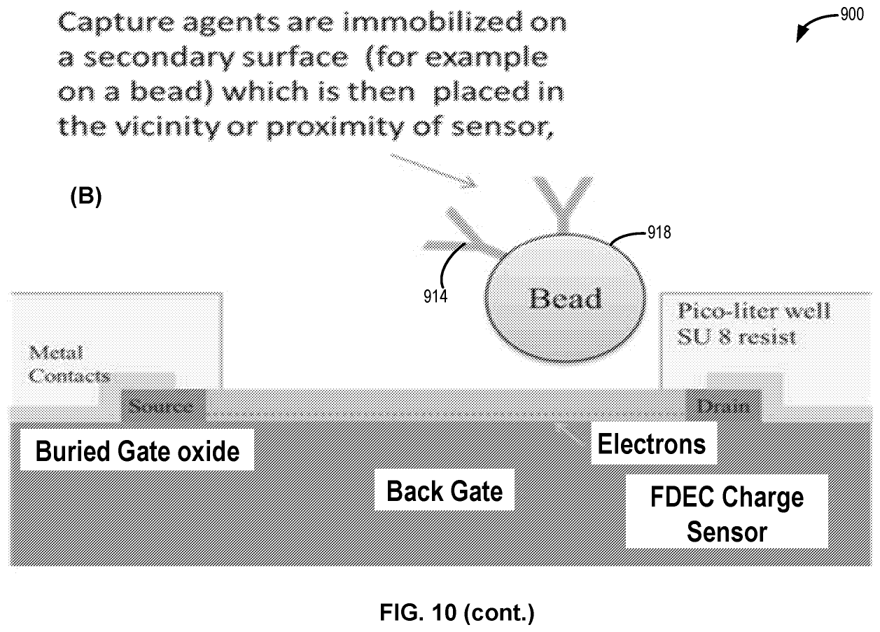

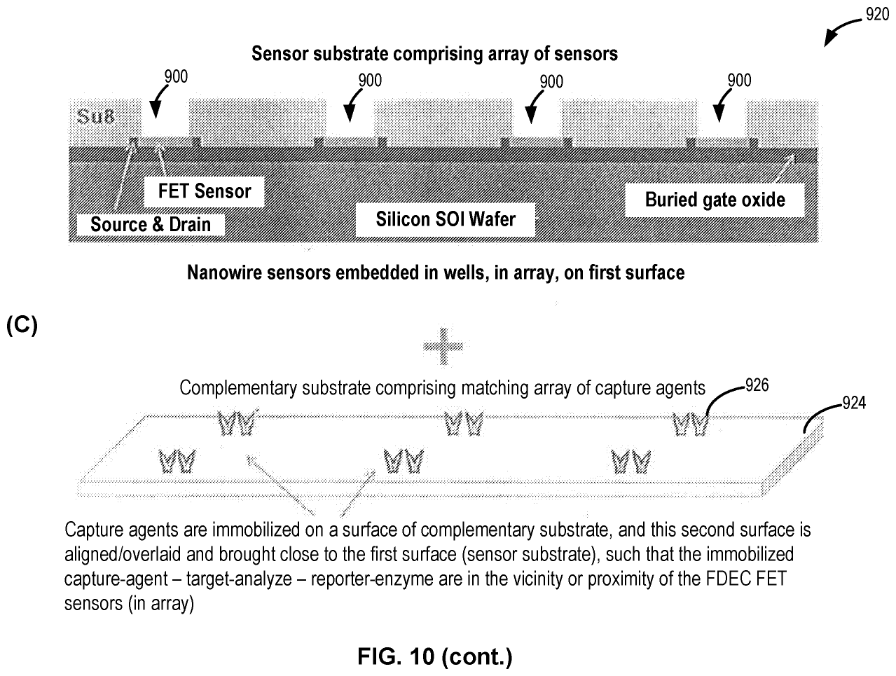

As illustrated in FIG. 10(A), a capture agent 914, 916 can be attached to a surface 920, 922 of sensor device 900. Additionally or alternatively, as illustrated in FIG. 10(B), a capture agent can be attached to a bead, which is place on or in the proximity to device 900. FIG. 10(C) illustrates that the sensor devices can form part of an array 920 formed on a substrate 922. Substrate 922 can be paired with a complimentary substrate 924 that includes a capture agent 926 and/or other molecules as described herein. Complimentary substrate 924 and substrate 922 can be aligned/overlaid and brought close to each other, such that the immobilized capture agent and target analyte, and a reporter enzyme are in the vicinity of sensor device 900.

2. Expose the Capture Agent to Test Sample. If Present, Target Molecule/Analyte in the Test Sample Binds to Capture Agent

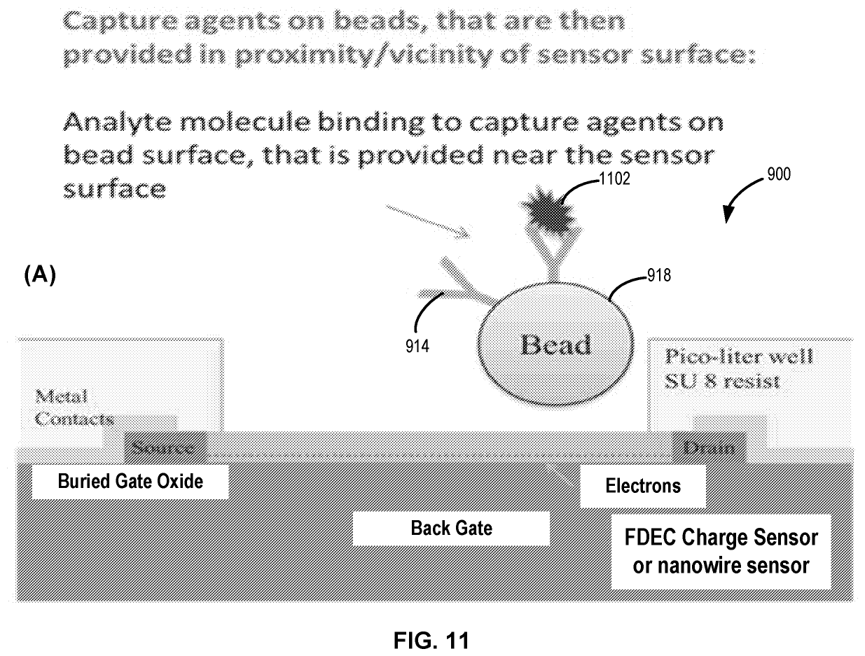

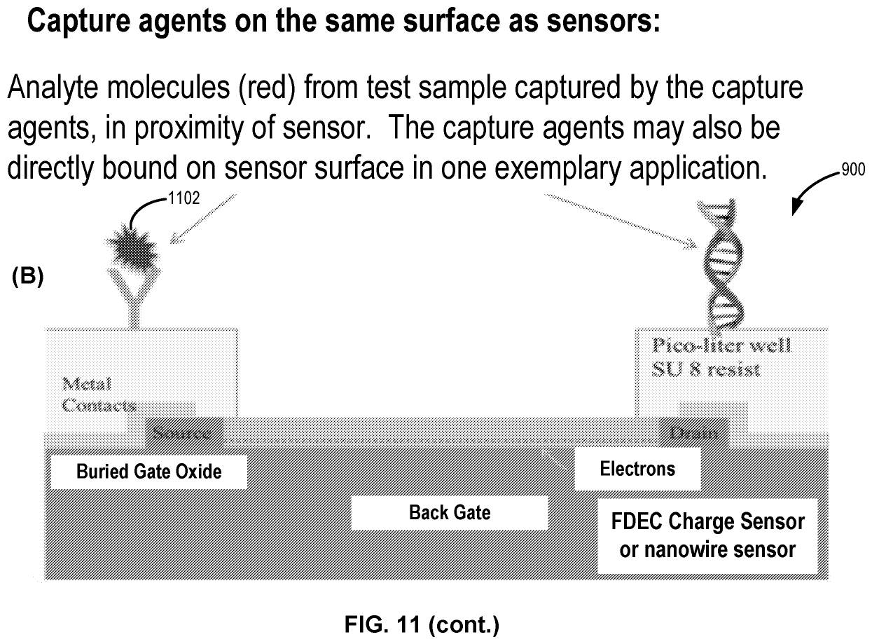

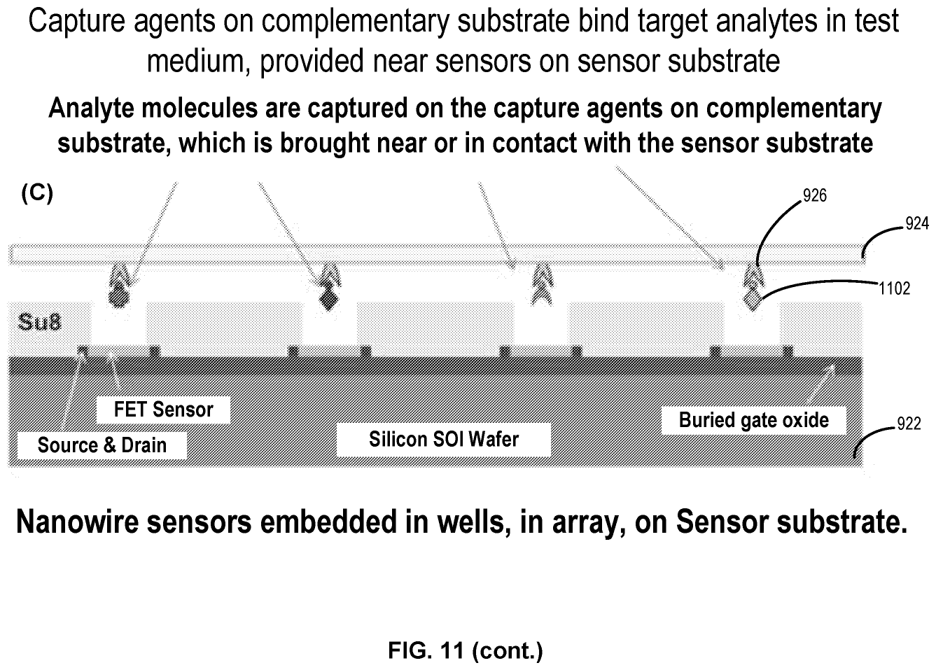

FIGS. 11(A)-11(C) illustrates a target analyte 1102 bound to various capture agents. In one non-limiting examples, capture agent binding to target analyte is specific and selective. Capture agent can be provided on same surface as the sensor or a different surface or bead 918 surface or other (e.g., immobilized) surface.

3. Introduce ‘complementary binding agent’ conjugated with ‘reporter enzyme’. Alternately introduce complementary binding agent and then introduce ‘Reporter Enzyme conjugated with tag’ where the ‘reporter tag’ binds to ‘complementary binding agent’

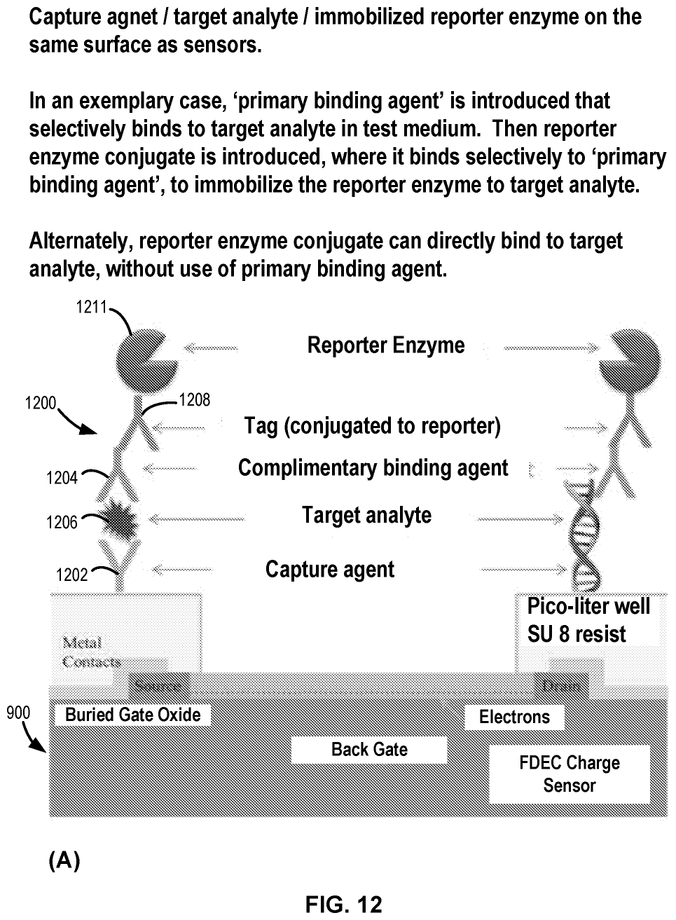

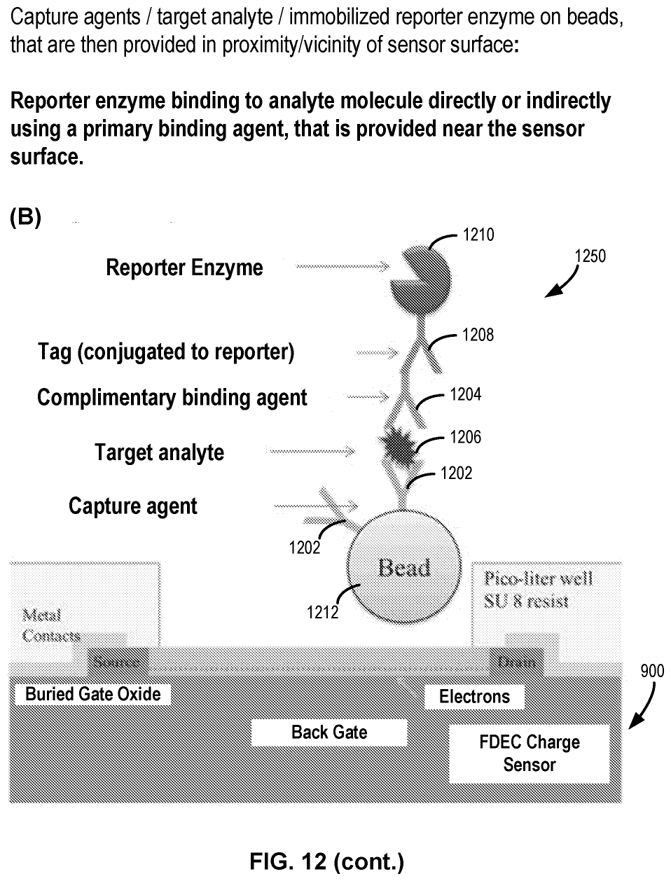

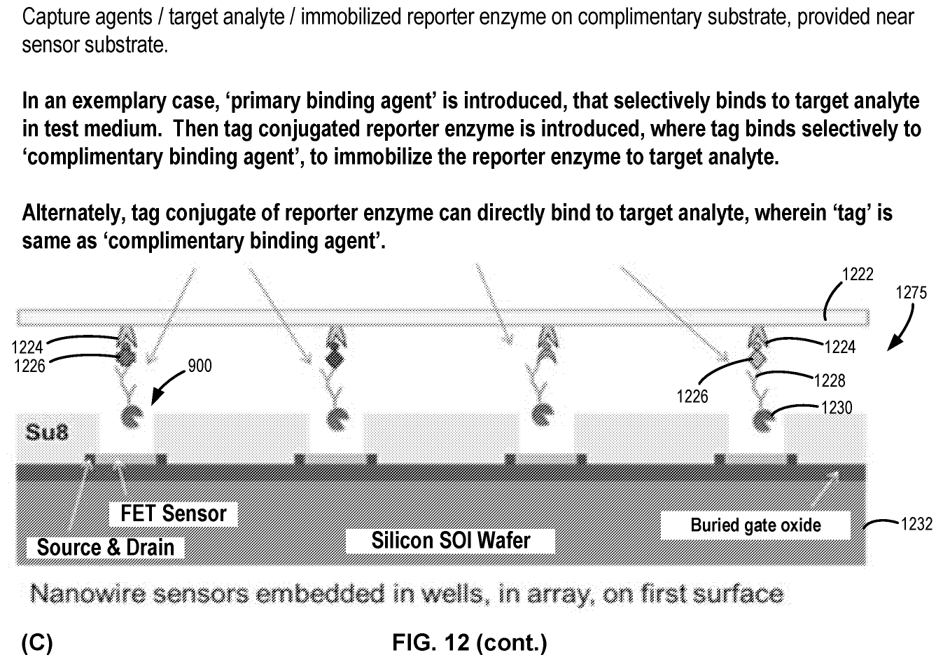

FIG. 12(A)-(C) illustrates a device 1200, 120, and 1275 with reporter enzyme conjugate that directly binds to target analyte, or indirectly binds to target analyte via primary binding agent in accordance with further exemplary embodiments of the disclosure. Devices 1200 and 1250 include sensor device 900, a capture agent 1202, a primary binding agent 1204 bound to a target analyte 1206, a conjugate biomolecule 1206, and a reporter enzyme 1208. The capture agent, target analyte, primary binding agent, conjugate molecule, and/or reporter enzyme can be the same as described elsewhere herein. Device 1250 additionally includes bead 1212 FIG. 12(C) illustrates a substrate 1222 to which a capture agent 1224, a target analyte 1226, a primary binding agent 1228, optionally a conjugate biomolecule, and a reporter enzyme 1230 are attached. Complimentary substrate 1222 and substrate 1232 can be aligned/overlaid and brought close to each other, as described above.
Non limiting examples are: Antibody conjugated reporter enzyme or complementary DNA/RNA/oligomer/binding-molecule conjugated reporter enzyme or reporter enzyme conjugated to molecule/species that binds or interacts selectively with target molecule is introduced, where the antibody or complementary molecule or DNA/RNA comprise the complimentary binding agent that binds to target analyte.
Conjugated reporter enzyme is immobilized/bound to the target analyte via one or both of ‘complementary binding agent’ and ‘tag.’ Target analyte has multiple binding epitopes, for binding with capture agent and for binding with ‘complementary binding agent’ or directly with ‘tag’.
Perform a wash to remove the un-immobilized test sample/substance and wash to remove un-immobilized conjugated reporter enzyme, in sequential steps as desired. Introduce enzyme substrate, wherein reporter enzyme catalyzes enzyme substrate turnover resulting in release of catalytic reaction products

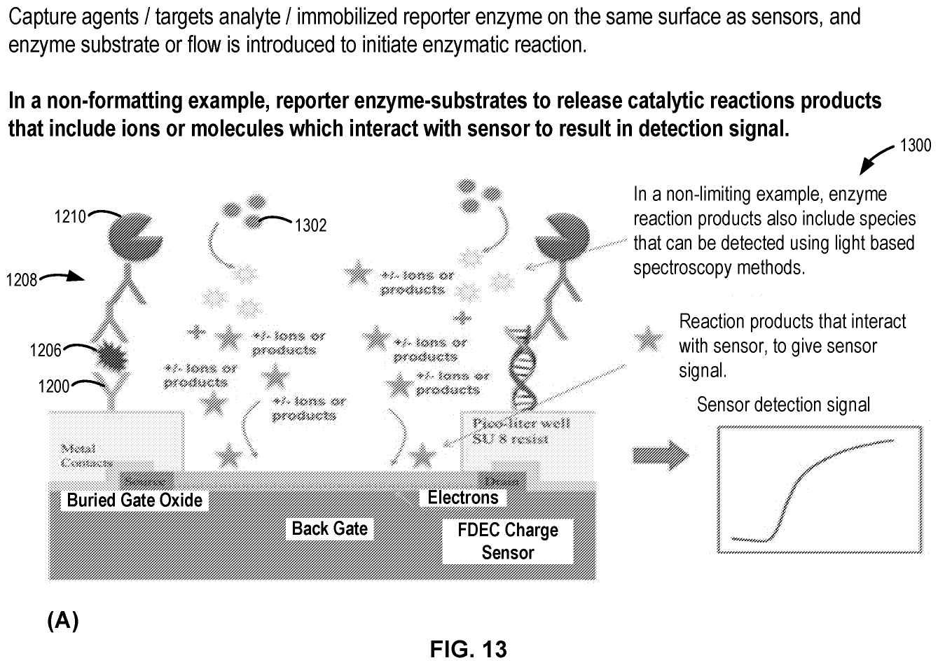

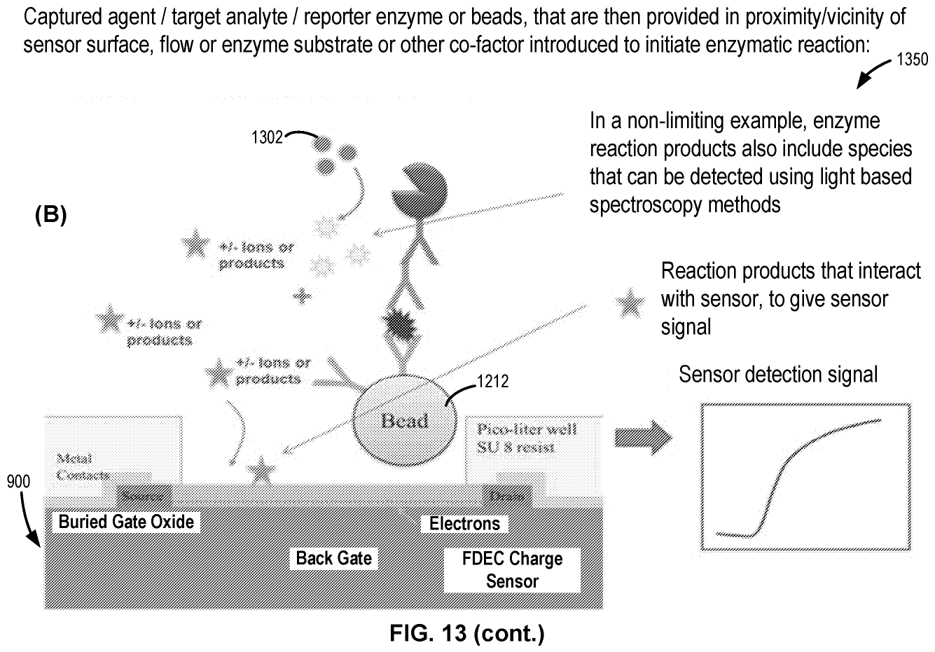

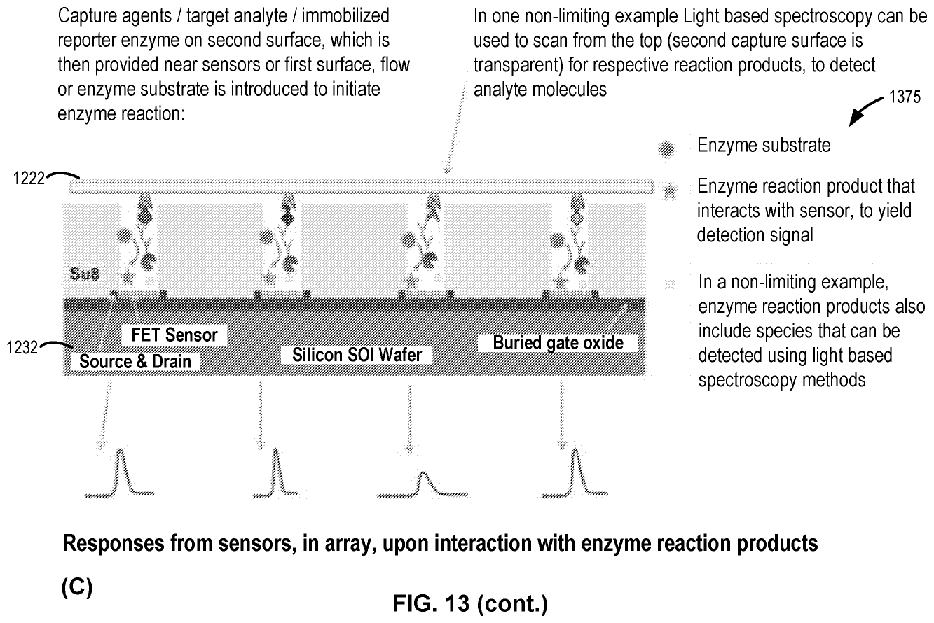

FIGS. 13(A)-(C) illustrates devices 1300, 1350, and 1375, which include the addition of an enzyme substrate 1302 that undergoes biochemical reaction in presence of reporter enzyme to produce enzyme reaction products,
Prior to introducing enzyme substrates, the sensor surface and assembly can be washed off any un-bound/un-immobilized/free reporter enzyme in solution.
Upon introducing, reporter enzyme interacts with the enzyme substrate 1302 in a specific reaction, releasing specific reaction products. The released enzyme reaction products can be ion species or non-ionic species, or other species.
In one exemplary case, reporter enzyme can auto-catalyze itself. A non-limiting example of this is auto-phosphorylating kinase enzymes in presence of ATP, Mg or Mn ions, and other kinase reaction components.
In one non-limiting example, the enzyme-substrate reaction products are localized near the sensor surface.
In one non-limiting exemplary case, the released reaction products are ions that are detected by the FDEC FET sensors or FET sensors.
In another non-limiting example, non-ionic reaction products of enzyme catalytic reaction are released which interact or bind to sensor surface, resulting in sensor response.
In another non-limiting example, enzyme reaction products are released which interact or bind to molecular or organic or inorganic monolayer or multiple layer film coated on sensor surface.
Interaction of enzyme reaction products with sensors yields a sensor signal.
Sensor signal from enzyme reaction products indicate the presence of analyte molecule, hence detecting the analyte molecule in high sensitivity.
Each enzyme can turnover a large number of substrate molecules, resulting in release of large number of reaction products, which interact with sensor to result in high signal to noise detection signal.
In one non-limiting exemplary case, a single reporter enzyme immobilized to a single analyte molecule can turn over a large number of enzyme substrates, resulting in high signal to noise detection signal.
In non-limiting exemplary cases, assay includes one or more blocking steps for preventing non-specific adhesion, after each of the binding/tagging steps.
Non-limiting examples of reporter enzymes can be phosphatases that release ions on catalysis, kinases that release H+ ions on catalysis, HRP that interacts with its substrate to release reaction products.
In one non-limiting example, when HRP is used as reporting enzyme, HRP substrate is chosen so that it releases an ion that interacts with the sensor to yield detection signal.
In non-limiting exemplary application, the reporter enzyme releases one or more products that can be detected by light based spectroscopy methods (fluorescence, luminescence, calorimetric detection, etc.) and by sensor devices in the vicinity, one after then other or simultaneously or in parallel, in the same assay.

The order of the above steps can be interchanged. And, in some exemplary cases not all or not each of the steps might be required and can be omitted. For example, the first step of providing the sensor can be done towards the end, wherein the capture agent is bound on a secondary surface or bead and then is brought into vicinity or proximity of the sensor surface.

In accordance with additional embodiments of the disclosure, devices and methods can be used to detect cell activity using enzyme linked sensors.

Enzyme Linked Transistor Amplified Activity (ELTAA) Assays: ELTAA assay technology is a non-optical, real-time electronic detection technology that offers potential for single molecule level sensitivity orders of magnitude higher sensitivity than ELISA. ELTAA assays combine exponential amplification inherent to enzyme—substrate interactions (enzyme linked) with high sensitive FDEC field effect transistor (FET) nanowire sensors (ultra sensitivity) for electronic signal readout.

As noted above, FDEC (fully depleted exponentially coupled) FET sensors can include nano-scale silicon transistor devices that can detect ions with ultra high sensitivity due to unique exponential charge coupling mechanism, wherein ions or molecules binding to FDEC nanowire sensor surface can switch the sensor device on/off—which is detected electronically as orders of magnitude change in transistor channel current. FDEC sensors are unique in that they directly detect charge variations at sensor surface. The direction of sensor response in FDEC sensors is opposite to that of generic ISFET or other FET sensors. Addition of H+ ions causes a decrease in threshold voltage of typical ISFET sensors, whereas in FDEC sensors H+ ion addition results in an increase in transistor threshold voltage. This is due to unique device physics that results in exponential capacitive charge coupling in FDEC sensors, effectively making them ultra-sensitive charge sensors or chemical reaction sensors. FDEC sensors can also be configured to detect potential or work function variations on sensor surface.

Enzyme—substrate interactions occurring in the vicinity/proximity of FDEC sensors (for example, within 100 micrometer distance), release ionic or molecular reaction products that may interact with sensor surface resulting in sensor response. ELTAA assays electronically detect enzyme—substrate functional activity (highly specific), in real-time (kinetic readout), using the integrated FDEC exponentially coupled charge sensors (ultra high sensitivity)—to indicate the presence of target-analyte or enzyme biomarkers in the test analyte.

Non Limiting Exemplary Embodiments of Enzyme Linked Sensor Assays for Cell Cycle Detection

Assays for cell death: Apoptosis (or programmed cell death) and necrosis are two forms of cell death that have been defined and well understood. While apoptosis is a physiological form of cell death that plays a critical role in the development and maintenance of multicellular organisms, necrosis is exclusively pathological, and does not confer any known benefit to the cell. A common factor in both mechanisms of cell death is the release of cellular contents. Similarly, cell proliferation biomarkers are often found on the surface of cell membrane or exuded into the growth medium. These cellular components have been exploited to develop cell death assays for diagnostic and drug discovery purposes. For example, cells undergoing apoptosis or necrosis exhibit internucleosomal DNA cleavage into oligonucleosome-length fragments which may be released outside the cell. DNA fragmentation, which is one of the hallmarks of cell death have been exploited for in situ measurement of cell death. Further, caspases and non-caspase proteases like cathepsins, calpains, and granzymes have also been implicated as effectors of apoptosis, and are found in exudates within the vicinity of apoptotic cells. Bacterial lysis and inhibition of cell wall formation are commonly associated with antibiotic treatment, cancer, and neurodegeneration. Specifically, most chemotherapeutic agents for cancer treatment effect disintegration of cancerous cells and several antibacterial agents cause necrosis of bacteria cells. Therefore, monitoring cell death using in vitro assays is essential to antibiotic and cancer drug development.

Current cell proliferation assay: cell proliferation assays include measurement of DNA synthesis, metabolic activity, antigens associated with cell proliferation, and ATP concentration. Metabolic cell proliferation assays rely on redox dyes e.g., tetrazolium salts and Alamar blue that are reduced in the environment of metabolically active cells. These assays also provide indirect measurement of cell death as previously described. Changes in the color of dye are monitored by spectrophotometer. ATP detection is another approach to measure cell proliferation because dying or dead cells contain little or no ATP. There is a linear relationship between cell proliferation and ATP concentration measured in a cell lysate. Cell proliferation can be measured by quantifying protein levels of key proliferation markers such as Ki-67, proliferating cell nuclear antigen (PCNA), topoisomerase IIB, phosphohistone H3, and minichromosome maintenance 2 (MCM 2).

Assay for detection of bacterial cell wall biosynthesis: variety of enzymes are present in the cell envelope of bacteria, within the periplasmic space and at the cell surface which are specifically released in the medium during cell growth or apoptosis. For example, alkaline phosphatase (ALP) is released into the medium by actively growing cells of Pseudomonas aeruginosa and Salmonella typhimuvium.

The enzyme sensor assays of present invention can be applied to detect cell growth, cell death, cell cycle, cell drug resistance, pathogen cell cycle, pathogen drug resistance by detecting/monitoring enzymes exuded/secreted from cells or pathogens or lysates in the presence of enzyme-substrates that are immobilized on or near surface of sensor device. Enzyme sensor arrays of current invention enable to quantitatively monitor cell proliferation and apoptosis in real-time.

Detection of cell activity using enzyme linked sensors: Cellular activity releases molecular and ionic species into extra cellular environment, where released species are specific to cellular activity such as growth, proliferation, division, death, apoptosis, necrosis, cell cycle etc. By detecting these specific molecules released by cells, it is possible to detect cell activity or viability or cell cycle; Enzymes are released by cells at various times in its growth and death cycles. Enzyme linked sensor assays of present invention can be used to detect the enzymes released to monitor cellular activity with high sensitivity

Enzyme Linked Sensor Assays (or ELTAA)

To detect ALP enzyme secretion, the enzyme-linked-sensor-assay for current incention can be used with commercial substrates of ALP such as 5-Bromo-4-Chloro-3-Indolyl Phosphate (BCIP), 4-Methylumbelliferyl phosphate (4-MUP), or p-Nitrophenyl Phosphate which may be immobilized on or near the sensor surface. The reaction products are ions or molecules that can be detected by sensor. As exemplary embodiment, ions are detected by FDEC FET sensors, to detect and monitor enzymatic activity.

Lactate dehydrogenase (LDH), a stable cytosolic enzyme, is currently one of the most common reporter enzymes of cell lysis. LDH released by apoptotic cells is assayed by incubating samples of culture broth with sodium pyruvate and NADH. LDH catalyzes reversible reduction of pyruvate to lactate in the presence of NADH. The reaction yields NAD+, which is measured spectrophotometrically at 340 nm. NADH oxidation can also be coupled to reduction of nitroblue tetrazolium to yield formazan, a chromophore, which is then quantified by colorimetry. NAD+ or hydrogen ions produced by LDH enzyme reaction can be monitored using sensor enzyme assay of current invention, and as exemplary embodiment, using FDEC FET sensors.

In cells, RNAP catalyzes transcription reaction that involves construction of RNA chain using DNA template. RNAP can be added to suitable buffer containing Mg²⁺ and ribonucleotides mix and exposed to sensor surface. The enzyme reaction produces hydrogen ions (H+) that can be detected as electrical signal by enzyme sensor assay of present invention. Because apoptotic cells exude DNA fragments into the medium, using RNAP as an exemplary embodiment, enzyme-sensor-assay for present invention can be used to detect exuding of DNA fragments, and hence detect cell apoptosis.

Another Non Limiting Exemplary Application: Proteome Array Based ELTAA Assays for Diagnosis and Prognosis of Diseases:

Cancer-ome on chip diagnostic or prognostic device for cancer: (i) produced by arraying all cancer related proteins on the chip (ii) including mutational versions of each of these cancer related proteins on the array (iii) optionally including post translationally modified (PTM) proteins (iv) and used for assaying the produced proteins, their mutational libraries with patient blood for immune response profiling (v) analyzing the signal from antibodies, immune cell and other immune response components binding to the antigenic proteins on the array, to determine the presence and absence of cancer, staging or grading of cancer, and/or to determine the effective therapeutic approach based on immune signature.

The proteins and their mutational versions can be from a type or sub-type of cancer, or multiple types of cancer, or all possible types of cancer. In similar fashion, the proteins and their mutations related to other diseases can be arrayed for diseaseome-on-chips for disease diagnosis and/or prognosis and/or effective therapy development.

The protein and protein mutational libraries can be produced on surface of biosensors where the immune signature of the binding antibodies, other immune components binding to antigenic proteins is detected by the biosensor response, for disease diagnosis and/or prognosis and/or effective therapy development. The enzyme linked sensor assay for present invention can be used in an array format to detect cancer, disease biomarkers.

Another Exemplary Application of Enzyme Linked Sensor Assays for Present Invention: Pathogen Identification/Detection and/or Antibiotic Susceptibility Testing System

A bacterial identification (ID) and antibiotic susceptibility testing (AST) system may comprise of sample to answer ID and AST system in few hours, comprising high detection sensitivity to assay few CFU, comprising phenotypic AST capable of assaying unknown bacteria (unknown genotypes) or new strains of known bacteria, potentially assaying un-culturable bacteria, capable of detecting hetero-resistance due to multiple resistant strains in a given sample, compact and portable, compatible with point of need (PoN) applications, automated for ease of use, high throughput system capable of comprehensive antibiotic susceptibility profiling against large panel of antibiotics, potentially real time data analysis that can be used for Biosurveillance.

Viable bacterial cells secrete or exude ATP/NADH dependent extra-cellular enzymes (exo-enzymes) such as phosphatases, proteases, lipases, esterases etc during growth and proliferation. Alkaline phosphatase (ALP) is one such enzyme, which plays role in cell wall biosynthesis. Similarly, upon cell death and lysis, cytoplasmic ATP/NADH dependent enzymes such as DNA gyrase, bacterial kinases, RNAP, MurA-F etc are released into medium. These enzyme classes need either ATP or NADP as co-factors for catalytic activity, and often result in charged ions as reaction products that can be detected by enzyme linked sensors of present invention. Detecting activity of secreted/exuded/leaked ion-producing enzymes can act as specific functional biomarkers of bacterial cell cycle. By printing (immobilizing) a unique substrate of an enzyme near a unique sensor, catalytic activity of even a single exo-enzyme on the printed substrate can be detected due to rapid substrate turnover and ion release. FDEC sensors are highly sensitive to ions, and to even partial charges. A flush of ions produced by single enzyme molecule (reporter-enzyme) turning over hundreds to million enzyme-substrates can readily be detected by FDEC sensors with high signal to noise. Signal from a sensor substrate pair indicates activity of corresponding specific enzyme in medium.

Enzyme linked sensor arrays of present invention can detect metabolomic and cell cycle biomarkers of bacterial isolates in antibiotic dilution titers to determine MIC. It can enable monitoring of bacterial susceptibility to antibiotics by detecting with high sensitivity (i) biomarkers of bacterial cell viability and proliferation such as alkaline phosphatase (ALP), proteases, lipases, esterases (ii) and biomarkers of bacterial cell death following bacterial cell lysis such as cytosolic enzymes DNA gyrase, bacterial kinases, MurA-F, RNAP among others. Assay can be performed with bacterial cells simultaneously with 50 antibiotics, with each antibiotic assayed at 20 different sequential dilutions, to determine MICs of 50 antibiotics, with comprehensive sample-to-answer anti-microbial susceptibility testing performed in few hours.

FIG. 14 illustrates a device 1400 that can be used to detect presence of specific enzymes that are released by cells during cellular activity and death, to indicate specific cellular activity or death, assayed by sensor detection of enzyme activity in presence of respective substrates that are immobilized in proximity of sensor surface. The assay can be performed in presence of putative drug molecules for drug discovery or in presence of drug molecules to detect drug resistance. Device 1400 includes sensor device 900, specific enzymes 1402, reporter enzymes 1404, and enzyme substrate 1406.

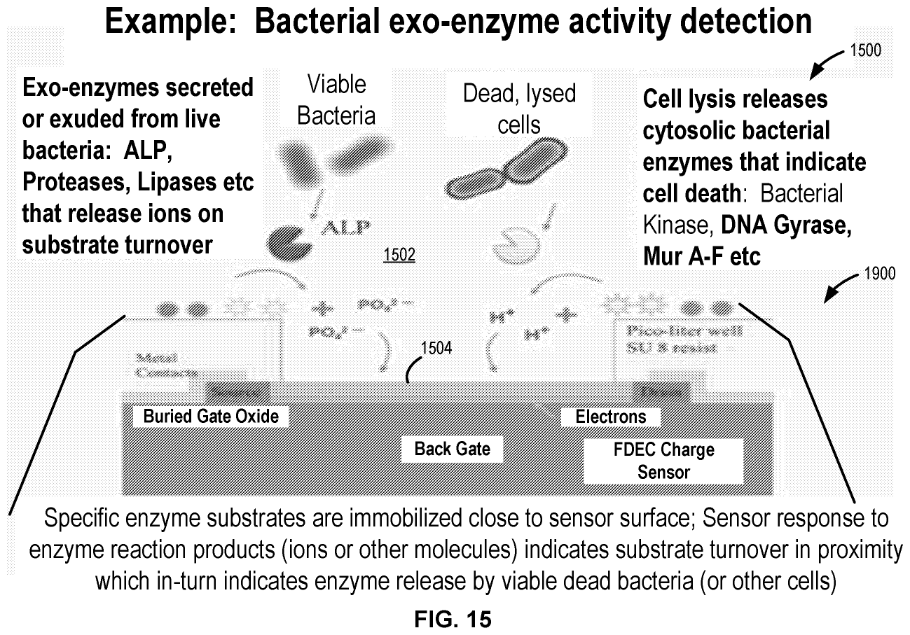

FIG. 15 illustrates a device 1500 in accordance with additional examples of the disclosure. Device 1500 can be used to detect cell death or growth or another cell cycle feature. Or alternately detect presence of specific cells or pathogens in the test medium. Enzyme-substrates to specific enzymes of interest can be immobilized on or in proximity of sensor surface. Enzyme substrates can alternately be immobilized on beads or another complementary substrate which can then be brought in proximity with the sensor surface. In presence of other reaction components, with enzyme-substrate immobilized in proximity, detected sensor signal implies presence of a specific enzyme, that catalyzes enzyme-substrate which resulted in sensor response. As a non-limiting example, live bacteria may secrete or exude enzymes such as phosphatases, ALP, proteases, lipases which may be detected by enzyme-linked sensor of present invention in presence of respective enzyme-substrate and other co-factors. As a non-limiting example, dead or lysed bacteria may secrete or exude enzymes such as bacterial kinases, Mur A-F enzymes, DNA gyrase, which may be detected by enzyme-linked sensor of present invention in presence of respective enzyme-substrate and other co-factors, buffers. In further exemplary application of enzyme linked sensor of present invention, the bacterial cell (or other pathogens) assay may be performed in the presence of drugs or antibiotics. Antibiotic resistance or drug resistance can be detected by monitoring for enzymes exuded/secreted by bacterial cell that are specific to cell growth and cell death or bacterial cell cycle.

Device 1500 includes a sensor device 900 that includes a surface 1504 in contact with an environment 1502 that includes, for example, vial bacterial and/or dea, lysed cells. In this case, specific enzyme substrates are immobilized close to surface 1504, which causes a change in electrical or other response of sensor device 900.

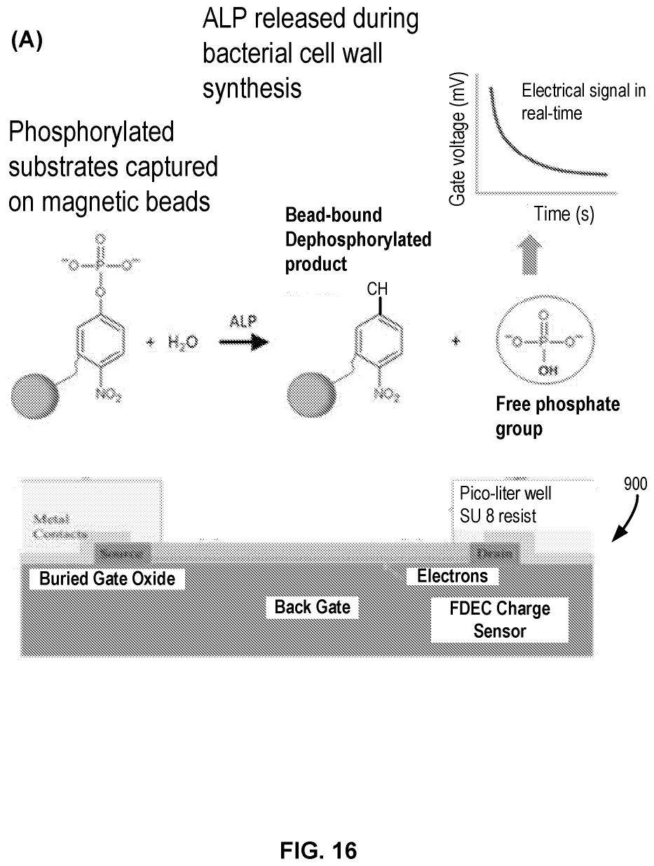

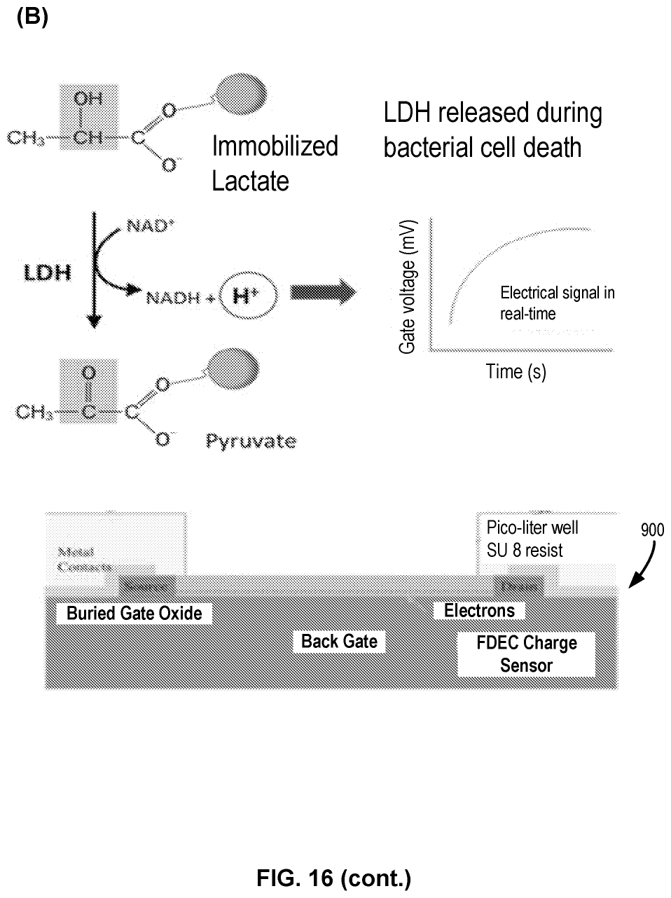

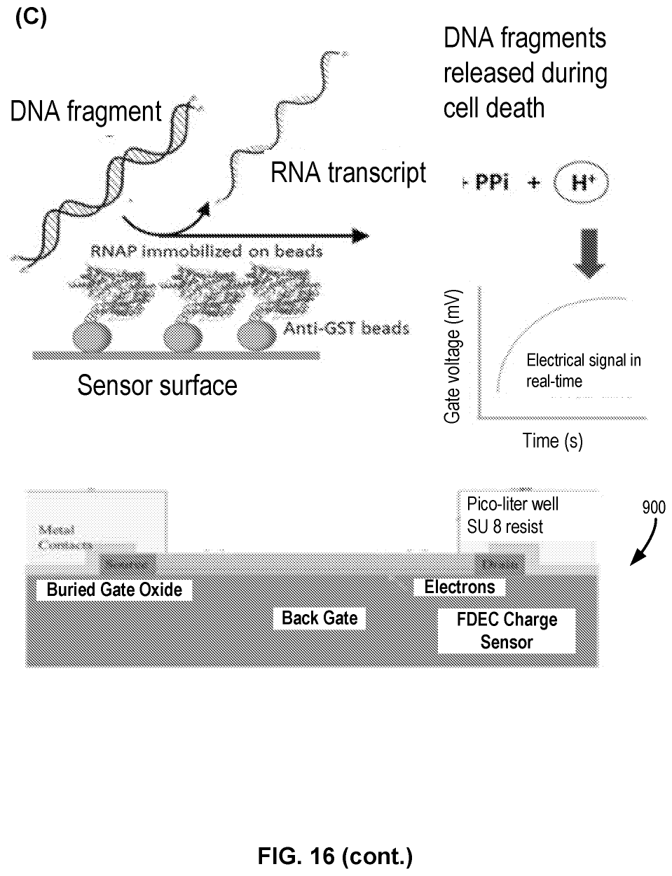

FIG. 16 illustrates example activity assays and cytotoxicity assays for detecting human and bacterial cells, using enzyme linked sensors of present invention. 16(A) illustrates an example of ALP phosphatase released by bacterial cells during cell wall synthesis. By immobilizing ALP substrate in proximity of enzyme linked sensor of present invention, ALP release by bacterial cell can be monitored, to detect bacterial cell growth. 16(B) illustrates as a non-limiting example, LDH (lactate dehydrogenase) enzyme is released by human cells at the time of stress or death, which can be detected using enzyme linked sensors of present invention in the presence of LDH substrate that in provided in vicinity of sensor. 16(C) illustrates as an example RNA polymerase (RNAP) enzyme linked sensor assay to detect DNA strands released by cells during cell cycle, cell communications, stress and death. In this exemplary case, RNAP enzyme is immobilized in proximity of sensor surface, which in the presence of other reaction components, transcribes any DNA strands released by the cells to RNA releasing H+ ions in addition to other reaction products. Enzyme linked sensor array detection of H+ ions can be used to monitor DNA secretion that informs on cell activity or cell state.

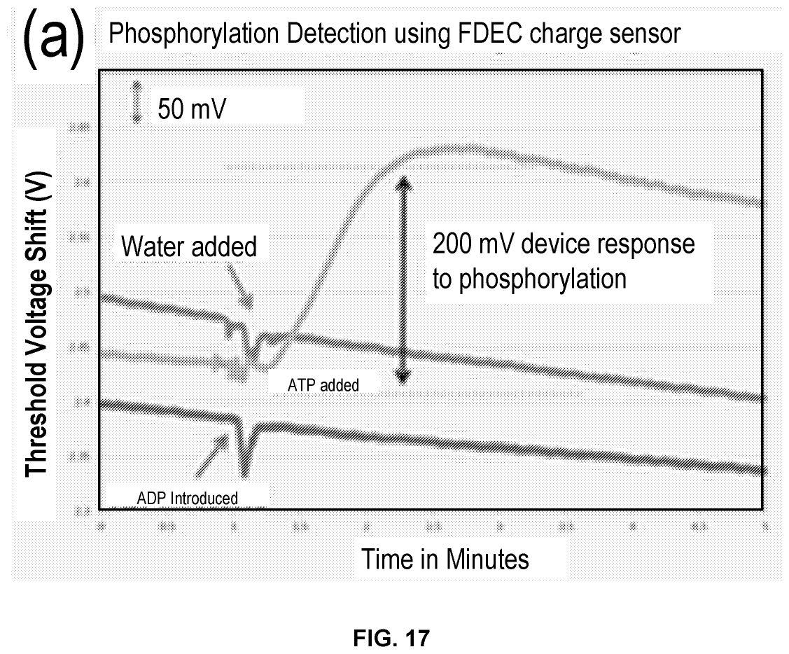

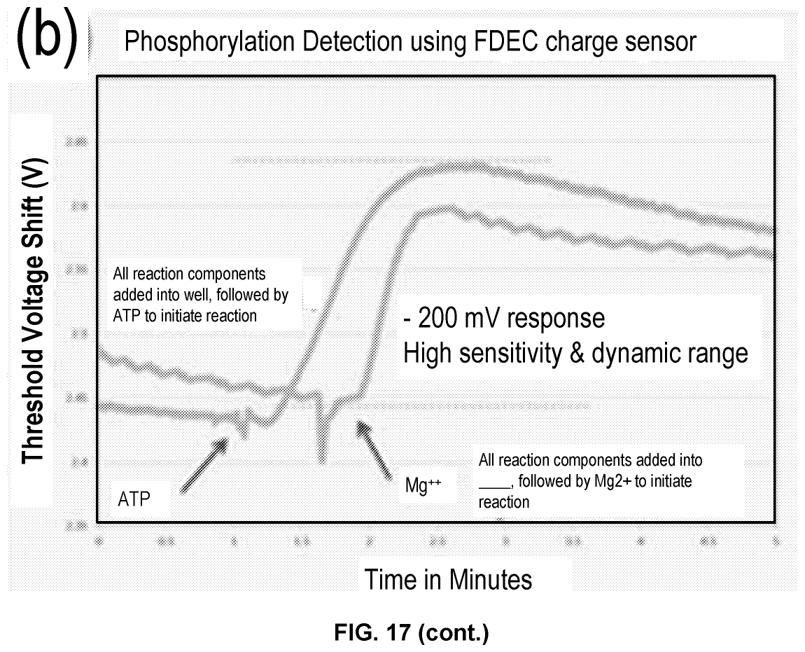

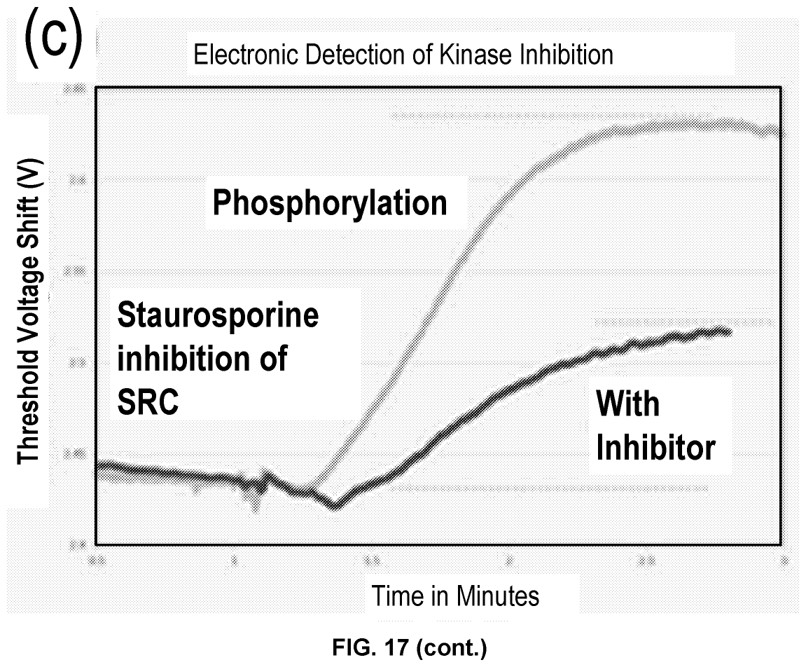

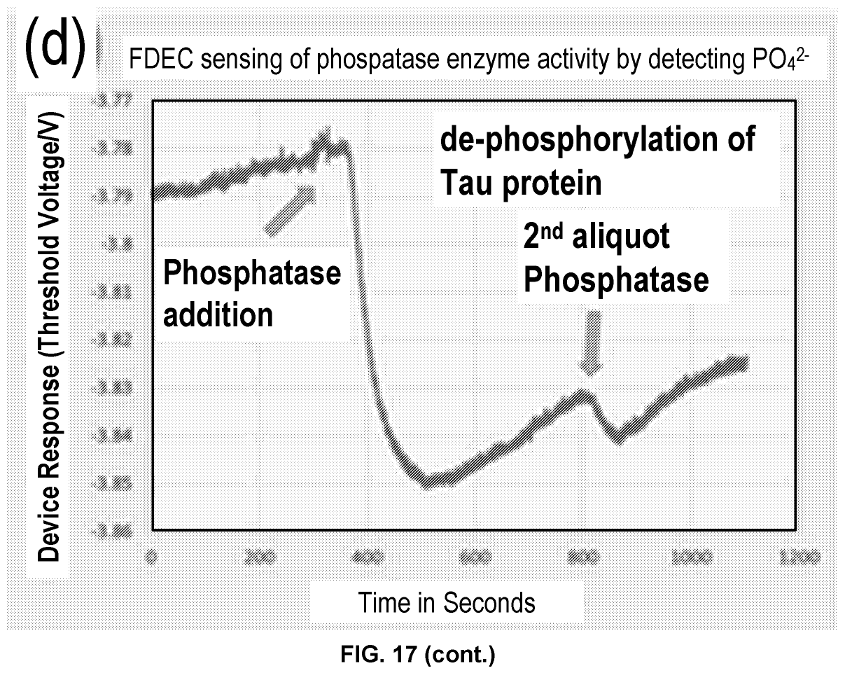

FIG. 17 illustrates exemplary sensor device signals detected in response to activity in an environment proximate the sensor device. FIGS. 17(A) and (B) illustrate FDEC sensor detection of kinase enzyme activity initiated by flow or addition of enzyme substrate or co-factor. FIG. 17(C) illustrates FDEC sensor detection of inhibition of kinase enzyme activity in the presence of a drug molecule, as example case of drug discovery screening or drug resistance detection application. FIG. 17(D) illustrates FDEC sensor detection of Tau enzyme-substrate de-phosphorylation by phosphatase enzyme.

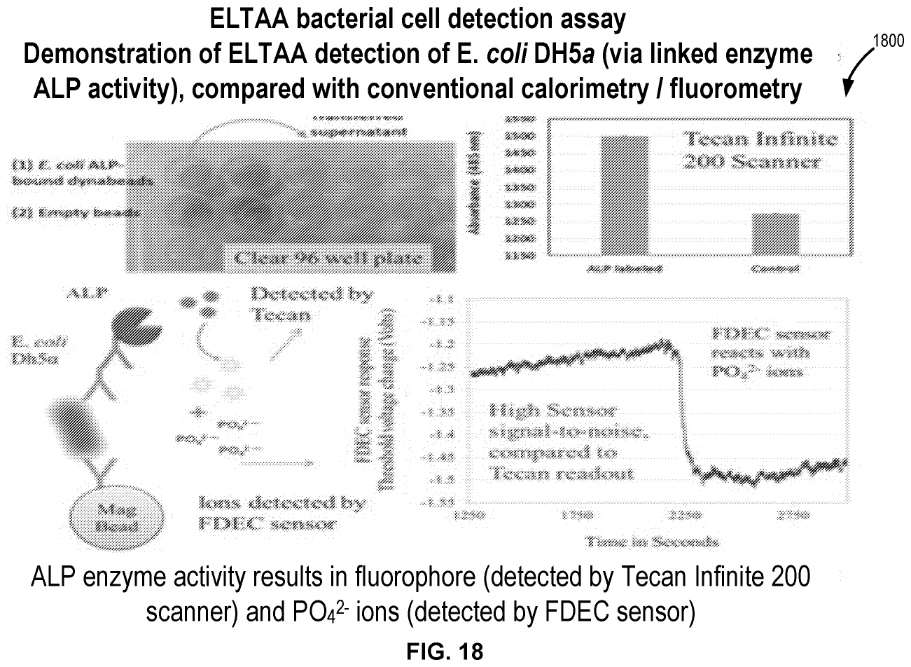

FIG. 18 illustrates a linked sensor assay 1800 (or ELTAA) according to exemplary embodiments of the disclosure. Linked sensor assay 1800 can be used to detect bacterial cell presence in a test medium, resulting in a sensor response. A magnetic bead coated with antibody can be used to capture any bacterial cells (test analyte) present in a test medium. Primary binding agent can be used to then bind to the bacterial cells (test analyte), followed by reporter enzyme conjugated to antibody. The beads can then introduced on to FDEC sensor device to present the analyte (bacterial cell) bound ALP reporter enzyme in proximity of sensor, and enzyme substrate was introduced to initiate ALP enzyme activity, in presence of other reaction components. Presence of bacterial cells in test medium was indicated by FDEC sensor response to ions produced by ALP enzyme reaction.

All publications and references referred to herein are incorporated here by reference in their entirely, to the extent the contents thereof do not conflict with the present disclosure.
Takulapalli, B. R. Molecular Sensing Using Monolayer Floating Gate, Fully Depleted SOI MOSFET Acting as an Exponential Transducer. Acs Nano 4, 999-1011 (2010).
Takulapalli, B. R. et al. Electrical detection of amine ligation to a metalloporphyrin via a hybrid SOI-MOSFET. Journal of the American Chemical Society 130, 2226-2233 (2008).
Takulapalli, B. R. et al. High Density Diffusion-Free Nanowell Arrays. Journal of Proteome Research 11, 4382-4391 (2012).
Patent: Quantum wire sensor and methods of forming and using same, U.S. Pat. No. 7,994,593 B2
Patent: Biomarker sensor array and circuit and methods of using and forming same, US 20160041155 A1
Patent: Nano structured field effect sensor and methods of forming and using same, U.S. Pat. No. 9,170,228 B2
Patent: Biosensor microarray compositions and methods, WO 2014143954 A2
Patent: Programmable arrays, WO 2013063126 A3
Paper: Single cell analytic tools for drug discovery and development, James R. Heath, Antoni Ribas, and Paul S. Mischel, Nat Rev Drug Discov. 2016 Mar.; 15(3): 204-216.
Thesis: Label-free and Multi-parametric Monitoring of Cell-based Assays with Substrate-embedded Sensors, DISSERTATION, Maximilian Oberleitner, 2015, University of Regensburg.

I claim:

1. A device for detecting a target analyte, the device comprising:
a sensor device comprising a surface exposed to an environment;
a capture agent on or near the surface, the capture agent configured to selectively bind to a target analyte;
optionally, a primary binding agent that binds to the target analyte;
a reporter enzyme that binds to the primary binding agent or to the target analyte; and
an enzyme substrate that undergoes biochemical reaction in presence of reporter enzyme to produce enzyme reaction products,
wherein the sensor device produces a signal by detecting a change in an electrical property of the environment or a mechanical property of the surface due to the production of the enzyme reaction products.

2. The device of claim 1, wherein the capture agent is immobilized on or near the surface.

3. The device of claim 1, wherein the target-analyte comprises a molecule or biomarker or ionic species in a test medium.

4. The device of claim 1, wherein the enzyme reaction products comprise ions.

5. The device of claim 1, wherein the enzyme reaction products bind to the surface.

6. The device of claim 1, wherein the electrical property comprises one or more of a charge and potential.

7. The device of claim 1, wherein the sensor device comprises a field effect transistor (FET) sensor or a fully depleted exponentially coupled (FDEC) FET sensor.

8. Device of claim 1, wherein the sensor device is present in an array format comprising a plurality of sensor devices, with a unique capture agent immobilized on or near each of the sensor devices.

9. Device of claim 1, wherein the sensor device is present in an array format comprising a plurality of sensor devices, with a unique capture agent immobilized on or near multiple sensors in the array.

10. A device for detecting a target analyte, the device comprising:
a sensor substrate comprising a sensor device;
a complimentary substrate comprising a capture agent to selectively bind to a target analyte;
optionally, a primary binding agent that binds to the target-analyte;
a reporter enzyme that binds to the primary binding agent or to the target analyte; and
an enzyme-substrate that undergoes biochemical reaction in presence of reporter enzyme to produce enzyme reaction products,
wherein the sensor device produces a signal by detecting a change in an electrical property of the environment or a mechanical property of the surface due to the production of the enzyme reaction products.

11. The device of claim 10, wherein the complimentary substrate comprises an array of capture-agents.

12. The device of claim 10, wherein the sensor substrate comprises an array of sensor devices.

13. The device of claim 10, wherein the sensor substrate comprising the array of sensor devices is aligned and overlaid with the complimentary substrate comprising a matching array of capture-agents, to create a microfluidic channel for fluidic flow between the sensor substrate and the complimentary substrate.

14. The device of claim 13, wherein the flow initiates a reporter enzyme reaction that results in enzyme-reaction-products.

15. The device of claim 10, wherein the complimentary substrate comprises multiple capture agents, with a unique capture agent immobilized at each spot of a plurality of spots.

16. The device of claim 10, wherein the complimentary substrate comprises epitope-tag fusion-proteins that are immobilized on surface using anti-epitope antibodies, wherein the fusion-proteins may be expressed in situ in an array format.

17. A method for detecting a target analyte, the method comprising the step of:
providing a sensor device comprising a surface;
providing a capture-agent on or near the surface;
exposing the surface to an environment comprising a target analyte;
optionally providing an primary binding agent that binds to the target analyte providing a reporter enzyme that binds to a primary binding agent or to the target analyte;
providing an enzyme-substrate that undergoes biochemical reaction in presence of the reporter enzyme to produce enzyme reaction products; and
using the sensor device, producing a signal by detecting a change in an electrical property of the environment or a mechanical property of the surface due to the production of the enzyme reaction products.

18. The method of claim 17, wherein the sensor device is a field effect transistor (FET) sensor or a fully depleted exponentially coupled (FDEC) FET sensor.

19. A device for detecting a target analyte, the device comprising:
a sensor device comprising a surface exposed to an environment;
a capture agent on or near the surface, the capture agent configured to selectively bind to a target analyte;
optionally, a primary binding agent that binds to the target-analyte;
an enzyme-substrate conjugate that binds to the primary binding agent or to the target analyte; and
an reporter-enzyme that undergoes biochemical reaction in presence of enzyme-substrate to produce enzyme reaction products,
wherein the sensor device produces a signal by detecting a change in an electrical property of the environment or a mechanical property of the surface due to the production of the enzyme reaction products.

20. The device of claim 19, wherein the enzyme-substrate comprises one or more of DNA strand, RNA strand, oligonucleotide strand, and the reporter-enzyme comprises one or more of, but not limited to, a polymerase or RNA polymerase or transcriptase or reverse transcriptase.

21. An enzyme linked sensor assay comprising:
a sensor device comprising a surface exposed to an environment;
optionally, a capture agent on or near the surface, the capture agent configured to bind one of either enzyme-substrate-conjugate or reporter-enzyme-conjugate that is a complementary one of enzyme-substrate-conjugate or reporter-enzyme-conjugate immobilized on or near the sensor surface, directly or using capture agent, the other of complementary reporter-enzyme or enzyme-substrate that undergoes biochemical reaction in presence immobilized conjugate to produce enzyme reaction products,
wherein the sensor device produces a signal by detecting a change in an electrical property of the environment or a mechanical property of the surface due to the production of the enzyme reaction products.

22. The enzyme linked sensor assay of claim 21, wherein the target analyte is a cell or pathogen in test medium that exudes the reporter-enzyme.

23. The enzyme linked sensor assay of claim 21, wherein the reporter-enzyme is exuded by a cell or pathogen.

24. The enzyme linked sensor assay of claim 21, wherein a drug molecule is added to the biochemical reaction to test for drug resistance.

25. The enzyme linked sensor assay of claim 21, wherein a putative drug molecule is added to biochemical reaction for screening or discovering new drugs.

26. The enzyme linked sensor assay of claim 21, wherein the reporter-enzyme or enzyme-substrate is isolated from diseased blood or tissue or saliva or other biosample from a patient.

27. The enzyme linked sensor assay of claim 21, wherein the reporter-enzyme or enzyme-substrate is isolated from pathogens.
